SEQUENCE LISTING (35 sequences)

SEQ ID NO: 1 - RNA, 20 nt, synthetic construct
  source 1..20: mol_type = other RNA
tgatcactggcaccatcact

SEQ ID NO: 2 - RNA, 20 nt, synthetic construct
  source 1..20: mol_type = other RNA
taaagccattcgccttccta

SEQ ID NO: 3 - RNA, 20 nt, synthetic construct
  source 1..20: mol_type = other RNA
aatgctctcaggtatggcgg

SEQ ID NO: 4 - RNA, 20 nt, synthetic construct
  source 1..20: mol_type = other RNA
attcctgagtaggcctgggt

SEQ ID NO: 5 - RNA, 20 nt, synthetic construct
  source 1..20: mol_type = other RNA
actttgtcaagctcatttcc

SEQ ID NO: 6 - RNA, 20 nt, synthetic construct
  source 1..20: mol_type = other RNA
cacagggtactttattgatg

SEQ ID NO: 7 - RNA, 20 nt, synthetic construct
  source 1..20: mol_type = other RNA
ctcgacaccagggcgttatg

SEQ ID NO: 8 - RNA, 21 nt, synthetic construct
  source 1..21: mol_type = other RNA
ccactccatgctcgataggat

SEQ ID NO: 9 - RNA, 17 nt, synthetic construct
  source 1..17: mol_type = other RNA
tgatggacgggtccggg

SEQ ID NO: 10 - RNA, 20 nt, synthetic construct
  source 1..20: mol_type = other RNA
caaaagggcccctgtcttca

SEQ ID NO: 11 - RNA, 20 nt, synthetic construct
  source 1..20: mol_type = other RNA
tgacacgcttccctggattg

SEQ ID NO: 12 - RNA, 20 nt, synthetic construct
  source 1..20: mol_type = other RNA
gctcgacgctaggatctgac

SEQ ID NO: 13 - RNA, 21 nt, synthetic construct
  source 1..21: mol_type = other RNA
aggatgaaatttggcatgggg

SEQ ID NO: 14 - RNA, 19 nt, synthetic construct
  source 1..19: mol_type = other RNA
tccctggggccacaaatct

SEQ ID NO: 15 - RNA, 20 nt, synthetic construct
  source 1..20: mol_type = other RNA
ccctccactcggaaggacta

SEQ ID NO: 16 - RNA, 20 nt, synthetic construct
  source 1..20: mol_type = other RNA
gctggtgcattttcggttgt

SEQ ID NO: 17 - RNA, 20 nt, synthetic construct
  source 1..20: mol_type = other RNA
aggcattgacaacagggttc

SEQ ID NO: 18 - RNA, 20 nt, synthetic construct
  source 1..20: mol_type = other RNA
gttgcacatcagcagcactt

SEQ ID NO: 19 - RNA, 20 nt, synthetic construct
  source 1..20: mol_type = other RNA
tggagagagtaaggcgcatc

SEQ ID NO: 20 - RNA, 20 nt, synthetic construct
  source 1..20: mol_type = other RNA
cctgggagtgcagggtttat

SEQ ID NO: 21 - RNA, 20 nt, synthetic construct
  source 1..20: mol_type = other RNA
atgacagcttggagcatctg

SEQ ID NO: 22 - RNA, 20 nt, synthetic construct
  source 1..20: mol_type = other RNA
tggagattttgccattttca

SEQ ID NO: 23 - RNA, 20 nt, synthetic construct
  source 1..20: mol_type = other RNA
taggaaggcgaatggcttta

SEQ ID NO: 24 - RNA, 20 nt, synthetic construct
  source 1..20: mol_type = other RNA
ccatggccacaagagttacc

SEQ ID NO: 25 - RNA, 19 nt, synthetic construct
  source 1..19: mol_type = other RNA
ctgccatgcagaggagaag

SEQ ID NO: 26 - RNA, 20 nt, synthetic construct
  source 1..20: mol_type = other RNA
tggcctttcctttatgcact

SEQ ID NO: 27 - RNA, 21 nt, synthetic construct
  source 1..21: mol_type = other RNA
gcaagatctcaccagaaacca

SEQ ID NO: 28 - RNA, 27 nt, synthetic construct
  source 1..27: mol_type = other RNA
cagtttattgtttcacagttctagagg

SEQ ID NO: 29 - RNA, 19 nt, synthetic construct
  source 1..19: mol_type = other RNA
tctcaccagaaaccacccc

SEQ ID NO: 30 - RNA, 20 nt, synthetic construct
  source 1..20: mol_type = other RNA
cagttcaggctgctgtaaca

SEQ ID NO: 31 - RNA, 16 nt, synthetic construct
  source 1..16: mol_type = other RNA
tggagattttgccatt

SEQ ID NO: 32 - RNA, 15 nt, synthetic construct
  source 1..15: mol_type = other RNA
aacacgtctatacgc

SEQ ID NO: 33 - RNA, 16 nt, synthetic construct
  source 1..16: mol_type = other RNA
gatgaactcgttacgc

SEQ ID NO: 34 - RNA, 150 nt, Homo sapiens
  source 1..150: mol_type = other RNA
ctccaaacctgtcccccacacccaccccacccccttgttgccaagttgtaagatcttgggctgacttccaagttcccaaaattcaagtgtggaaaaagccaactaatctcctggaaaaatctcctgatgtgtatttttatttaacaatct

SEQ ID NO: 35 - RNA, 16 nt, synthetic construct
  source 1..16: mol_type = other RNA
ctgatgcgccttactc

(19) United States
(12) Patent Application Publication
(10) Pub. No.: US 2025/0277213 A1
(43) Pub. Date: Sep. 4, 2025

(54) COMPOSITIONS AND METHODS RELATED TO MULTIPLE MYELOMA-ASSOCIATED LONG NONCODING RNAS

(71) Applicant: Washington University, St. Louis, MO (US)

(72) Inventor: Jessica Silva-Fisher, St. Louis, MO (US)

(73) Assignee: Washington University, St. Louis, MO (US)

(21) Appl. No.: 19/066,008

(22) Filed: Feb. 27, 2025

Related U.S. Application Data
(60) Provisional application No. 63/559,613, filed on Feb. 29, 2024.

(51) Int. Cl. C12N 15/113; A61K 31/198; A61P 35/00; C12Q 1/6886

(52) U.S. Cl. CPC C12N 15/113; A61K 31/198; A61P 35/00; C12Q 1/6886; C12N 2310/11; C12N 2310/3231; C12N 2310/341; C12Q 2600/158

(57) ABSTRACT

Among the various aspects of the present disclosure are provisions of compositions and methods related to multiple myeloma-associated long noncoding RNAs. The present disclosure describes a composition that comprises a long non-coding RNA (lncRNA) inhibitor targeting LINC01432. Also disclosed is a method to select a treatment for a multiple myeloma patient, as well as to treat a multiple myeloma patient, both related to targeting LINC01432.

### CROSS-REFERENCE TO RELATED APPLICATIONS

This application claims the benefit of priority to U.S. Provisional Application Ser. No. 63/559,613 filed on Feb. 29, 2024, which is incorporated herein by reference in its entirety.

### STATEMENT REGARDING FEDERALLY SPONSORED RESEARCH OR DEVELOPMENT

Not applicable.

### MATERIAL INCORPORATED-BY-REFERENCE

The Sequence Listing, which is a part of the present disclosure, includes a computer-readable form comprising nucleotide and/or amino acid sequences of the present invention (file name “020751-US_NP_squence_listing” created on 26 Feb. 2025; 31,831 bytes). The subject matter of the Sequence Listing is incorporated herein by reference in its entirety.

The RNA sequencing data, which is a part of the present disclosure, is available at GEO under accession number GSE267013 and is incorporated herein by reference in its entirety.

### FIELD OF THE INVENTION

The present disclosure generally relates to compositions and methods related to the treatment of multiple myeloma.

### BACKGROUND OF THE INVENTION

Multiple myeloma is one the most common hematologic malignancies that accounts for about 13% of all hematologic malignancies and 1% of overall cancer. Although survival and response to the standard care of treatment using high dose melphalan followed by autologous stem cell transplant have improved prolonged event-free survival, the overall survival remains dismal. The occurrence of drug resistant, lack of selectivity, and high toxicity are the primary limiting factors for the long-term success of this treatment thereby most patients suffer a fatal relapse. In addition, interpatient heterogeneity, use of microarrays, and bulk RNA sequencing have further prevented the identification of the molecular mechanisms that control the malignant progression of myeloma plasma cells. Thereby, the lack of a basic understanding of mechanisms and reliable biomarkers to predict which myeloma patients will not respond to standard care of therapy and relapse is a critical barrier.

Many studies have focused on the 2% of genes in the human genome that are protein-coding genes, but our proposal will analyze long non-coding RNAs (lncRNAs) that are a type of RNA within the remaining 98% of the human genome that is often ignored. lncRNAs are greater than 200 nucleotides in length, do not encode proteins, and have a diverse range of epigenetic and biological functions, including serving in many functions associated with carcinogenesis and metastasis. The GENCODE consortium and others have estimated there are 15,000-23,000 unique lncRNAs, however many are not yet characterized. Yet, the diverse important critical biological functions including lncRNA-mediated gene regulation and lncRNA-protein binding that have proven to play important roles in promoting tumorigenesis and metastasis have begun to be uncovered. Significant contributions have identified novel lncRNAs in multiple solid tumor types including metastatic colon cancer. Further, research has provided insight into the importance of lncRNA deregulation in cancer and its association with drug resistance. More recently, deregulated lncRNAs associated with late-stage relapse breast cancer have been identified. Due to lncRNAs being highly tissue-specific they also show promise as novel treatments, prognostic, and diagnostic biomarkers.

### SUMMARY OF THE INVENTION

Among the various aspects of the present disclosure are provisions of compositions and methods related to the treatment of multiple myeloma.

Briefly, therefore, the present disclosure is directed to long non-coding RNAs associated with multiple myeloma and related compositions and methods of use thereof.

In one aspect, a composition for treatment of multiple myeloma in a patient in need is disclosed that includes a LINC01432 inhibitor. In some aspects, the LINC01432 inhibitor includes an antisense oligonucleotide (ASO). In some aspects, the ASO includes an in vitro-locked nucleic acid GapmeR antisense oligonucleotide (LNA ASO). In some aspects, the LNA ASO includes SEQ_ID_NO:31. In some aspects, the composition further includes a multiple myeloma chemotherapy. In some aspects, the chemotherapy is Melphalan. In some aspects, the composition further includes a CELF2 inhibitor. In some aspects, the CELF2 inhibitor is a CELF2 LNA ASO.

In another aspect, a method to treat multiple myeloma in a patient in need is disclosed that includes administering a therapeutically effective amount of a LINC01432 inhibitor. In some aspects, the LINC01432 inhibitor includes an antisense oligonucleotide (ASO). In some aspects, the ASO includes an in vitro-locked nucleic acid GapmeR antisense oligonucleotide (LNA ASO). In some aspects, the LNA ASO includes SEQ_ID_NO:31 or SEQ_ID_NO:35. In some aspects, the method further includes administering a therapeutically effective amount a multiple myeloma chemotherapy. In some aspects, the multiple myeloma chemotherapy includes Melphalan. In some aspects, the method further includes administering a therapeutically effective amount of a CELF2 inhibitor. In some aspects, the CELF2 inhibitor is a CELF2 LNA ASO.

In an additional aspect, a method of selecting a treatment for multiple myeloma in a patient in need is disclosed that includes quantifying an expression of a long non-coding RNA (lncRNA) comprising LINC01432, determining an expression level of the lncRNA, and selecting the treatment based on the expression level of the lncRNA. The selection of the treatment includes administering a therapeutically effective amount of a multiple myeloma chemotherapy if the expression level of the lncRNA is below a threshold value or administering the therapeutically effective amount of the multiple myeloma chemotherapy and a therapeutically effective amount of a LINC01432 inhibitor if the lcRNA expression is above the threshold value. In some aspects, the LINC01432 inhibitor includes an in vitro-locked nucleic acid GapmeR antisense oligonucleotide (LNA ASO). In some aspects, the LNA ASO includes SEQ_ID_NO:31 or SEQ_ID_NO:35. IN some aspects, the threshold level comprises a level about 6.42 fold higher than a healthy control expression level. In some aspects, the multiple myeloma chemotherapy includes Melphalan.

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1A is a schematic of the pipeline used to identify long non-coding RNAs (lncRNAs) associated with poor response to standard multiple myeloma (MM) therapy.

FIG. 1B is a volcano plot identifying significantly differentially expressed lncRNA (red) in poor responders, as compared to standard responders.

FIG. 1C is a pathway analysis of differentially expressed genes associated with poor responders.

FIG. 2A is a graph showing the expression of the lncRNA LINC01432 which has higher expression in poor responders compared to standard responders.

FIG. 2B is a graph showing the expression correlation of the poor response gene, LINC01432, and known MM translocations.

FIG. 2C is a set of fluorescent images of mFISH showing localized expression of LINC01432 (middle; red) in bone marrow aspirates of myeloma patients.

FIG. 2D is a set of fluorescent images of mFISH showing localized expression of LINC01432 (red) in the MM cells lines RPMI 8226 (top) and U266B1 (bottom). MM cells lines injected into mice subcutaneously. High magnification (right) of merged cells shows DAPI (blue) and LINC01432 (red) cells.

FIG. 2E is a graph quantifying nuclear and cytoplasmic co-localization of LINC01432 expression in RPMI 8226 from FIG. 2D, top.

FIG. 2F is a graph quantifying nuclear and cytoplasmic co-localization of LINC01432 expression in U266B1 from FIG. 2D, bottom.

FIG. 2G is a UMAP graph from single-cell RNA sequencing (scRNA seq) data showing expression of LINC01432 in normal subjects.

FIG. 2H is a UMAP graph from scRNA seq data showing expression of LINC01432 in newly diagnosed MM patients.

FIG. 2I is a UMAP graph from scRNA seq data showing cell populations in newly diagnosed MM patients.

FIG. 3A is a graph showing the expression of LINC01432 in RPMI 8226 cells with a CRISPR control (left) and CRISPR/Cas9-mediated LINC01432 knockdown (right).

FIG. 3B is a graph of control and LINC01432 knockdown RPMI 8226 cells showing cell viability (circle; black), cytotoxicity (square; blue), and apoptosis (triangle; red) as measured via ApoTox-Glo assay. *p=<0.05, #p=<0.0005.

FIG. 3C is a graph annexin V expression measuring apoptosis in CRISPR control (left) and LINC01432 knockdown (right) cells via Annexin V staining flow cytometry. #p=<0.0005.

FIG. 3D is a graph quantifying LINC01432 overexpression in U266B1 cells (right) as compared to empty vector control (left).

FIG. 3E is a graph of control and LINC01432 overexpression U266B1 cells showing cell viability (circle; black), cytotoxicity (square; blue), and apoptosis (triangle; red) as measured via ApoTox-Glo assay. *p=<0.05.

FIG. 3F is a graph annexin V expression measuring apoptosis in empty vector control (left) and LINC01432 overexpression (right) via Annexin V staining flow cytometry. *p=<0.05.

FIG. 3G is a graph quantifying tumor volume following subcutaneous injection of control CRISPER (solid line) and LINC01432 CRISPER knockdown (dash line) cells in NSG mice. *p=<0.05

FIG. 3H is a representative image of tumor growth of control CRISPR (left) and LINC01432 CRISPER knockdown (right) cells 42 days after subcutaneous injection into NSG mice.

FIG. 3I is a graph quantifying tumor volume following subcutaneous injection of empty vector control (solid line) and LINC01432 overexpression (dash line) cells in NSG mice. *p=<0.05, **p=<0.005, #p=<0.0005.

FIG. 3J is a representative image of tumor growth of empty vector control (left) and LINC01432 overexpression (right) cells 35 days after subcutaneous injection into NSG mice.

FIG. 4A is a schematic of POSTAR3 prediction of CELF2 binding to LINC01432 lncRNA.

FIG. 4B is an image of predicted binding sites of CELF2 on LINC01432 by POSTAR3.

FIG. 4C is a graph showing the expression of CELF2 in poor responders compared to standard responders, based on analysis of patient RNA sequencing data.

FIG. 4D is a set of fluorescent images showing the localization of LINC01432 lncRNA (red) and CELF2 protein (green) as determined by mFISH assay using LINC01432 RNA probes combined with CELF2 antibodies using immunohistochemistry in RPMI 8226 cells. Scale bare=40 μM.

FIG. 4E is a set of fluorescent images showing the localization of LINC01432 lncRNA (red) and CELF2 protein (green) as determined by mFISH assay using LINC01432 RNA probes combined with CELF2 antibodies using immunohistochemistry in U266B1 empty vector (top) and U266B1 LINC01432 overexpressing cells. Scale bare=40 μM.

FIG. 4F is a graph quantifying nuclear (red) and cytoplasmic (blue) localization of CELF2 and LINC01432 in RPMI 8226 cells using QuPath.

FIG. 4G is a graph quantifying nuclear (red) and cytoplasmic (blue) localization of CELF2 and LINC01432 in U266B1 empty vector control cells and LINC01432 overexpression cells using QuPath.

FIG. 4H is a schematic of in vitro of individual-nucleotide resolution UV crosslinking and immunoprecipitation (iCLIP) protocol.

FIG. 4I is a schematic of tiled primers used to identify prospective CELF2 binding site on LINC01432.

FIG. 4J is a graph of CELF2 iCLIP RT-qPCR data, showing CELF2 (black) binding to LINC01432 compared to IgG (grey) negative control. Actin and GAPDH serve as negative gene controls.

FIG. 5A is a graph showing the expression of LINC01432 in RPMI 8226 cells treated with Control LNA ASO or LINC01432-targeted LNA ASO, quantified by RT-qPCR.

FIG. 5B is a graph of control and LNA ASO-mediated LINC01432 knockdown in RPMI 8226 cells showing cell viability (circle; black), cytotoxicity (square; blue), and apoptosis (triangle; red) as measured via ApoTox-Glo assay.

FIG. 5C is a graph of combined treatment of RPMI 8226 cells with LINC01432 LNA ASO and Melphalan on apoptosis, as measured via Annexin V and propidium iodide (PI) flow cytometry. Live cells (grey), early apoptosis (blue), late apoptosis (red), necrosis (orange). *p<0.05, **p<0.005.

FIG. 5D is a graph of combined treatment of RPMI 8226 cells with LINC01432 LNA ASO (red) and Melphalan on proliferation, as compared to control LNA ASO (black), measured via IC50 assay. **p<0.005.

FIG. 6 is a schematic of overall outcomes in MM poor responders. LINC01432 is highly expressed in newly diagnosed multiple myeloma patients, is bound by CELF2 protein, and together they inhibit apoptosis and promote cell viability.

FIG. 7A is a gene ontology graph from scRNA seq LINC01432 data showing an enrichment score for gene sets related to biological processes (left; orange), cellular components (middle; green), and molecular functions (right; blue).

FIG. 7B is a visual representation of pathway analysis of genes associated with poor responders from scRNA seq LINC01432 data.

FIG. 8A is a graph showing LINC01432 expression in the MM cell lines RPM18226, OPM-2, MM.1R, MM.1S, and U266B1.

FIG. 8B is a graph showing the expression of LINC01432 when treated with control or Melphalan (10 μM and 30 μM).

FIG. 8C is a graph of LINC01432 expression in U266 cells when treated with melphalan (30 μM; orange), Dexamethazone (DEX; 10 μM; blue), Bortezomib (BTZ; 0.002 μM; red), and combo (BTZ+DEZ; green), as compared to control (black).

FIG. 8D is a schematic of tumor growth studies of control and LINC00XPRR CRISPR or overexpressing cells subcutaneously injected into nod scid gamma (NSG) mice.

FIG. 9A is a graph showing expression of TP53 pathway genes (LINC01432, p53, cMYC, BAX) in control and LINC01432 knockdown cells.

FIG. 9B is a graph showing expression of TP53 pathway genes (LINC01432, p53, cMYC, BAX) in empty vector and LINC01432 overexpression cells.

FIG. 9C is a representative image of a western blot showing a decrease in γH2AX in LINC01432 overexpressed cells (right) compared to empty vector (left).

FIG. 10 is a schematic of LINC01432 interaction network displayed using POSTAR3 program which identified binding to CELF2 and AGO2 proteins.

FIG. 11A is a graph showing the expression of CELF2 in different blood cancers from Cancer Cell Line Encyclopedia.

FIG. 11B is a western blot showing CELF2 (top) expression in control cells and LINC01432 CRISPER knockdown cells as compared to the reference gene, tubulin (bottom).

FIG. 11C is a western blot showing CELF2 (top) expression in control cells and LINC01432 overexpressing cells as compared to the reference gene, tubulin (bottom).

FIG. 11D is a schematic of CLEF2 binding site prediction (red) on LINC01432.

FIG. 11E is a graph showing the expression of CELF2 on different blood cell types.

FIG. 12A is a graph of CELF2 expression in U266B1 cells treated with control locked nucleic acid oligonucleotides (LNA ASO), or CELF2 LNA ASO1, or CELF2 LNA ASO4.

FIG. 12B is a graph of apoptosis induced by U266B1 cells treated with LNA ASO, CELF2 LNA ASO1, or CELF2 LNA ASO4.

FIG. 13A is a schematic of identification of deregulated lncRNAs in newly diagnosed multiple myeloma patients associated with high does melphalan (HDM) autologous stem cell transplant (ASCT) 3-drug treatment.

FIG. 13B is a gene set enrichment analysis (GSEA) of genes associated with poor responders.

FIG. 14A is a set of multiplexed fluorescent RNA In situ Hybridization (mFISH) images using LINC01432 (red) probes in RPM18226 cells.

FIG. 14B is a set of mFISH images using LINC01432 (red) RNA probes in MM patient bone marrow.

FIG. 14C is a set of mFISH images using LINC01432 (red) RNA probes in U266B1 control (top), U266B1 Melphalan treated (24 h; middle), and U266B1 Melphalan treated (72 h; bottom) cells.

FIG. 14D is a graph quantifying the fluorescence of LINC01432 (red) RNA probes in U266B1 control (top), U266B1 Melphalan treated (24 h; middle), and U266B1 Melphalan treated (72 h; bottom) cells, as seen in FIG. 14C.

FIG. 15A is a graph of LINC01432 expression in cells treated with control or LINC01432 LNA ASO1, LNA ASO2, or LNA ASO3.

FIG. 15B is a graph of MM.1R cells treated with control LNA ASO or LNA ASO1 showing cell viability (circle; black), cytotoxicity (square; blue), and apoptosis (triangle; red) as measured via ApoTox-Glo assay.

FIG. 15C is a set of flow cytometry plots showing annexin V staining in cells treated with control LNA (left) and LNA ASO1 (right).

FIG. 15D is a graph quantifying the annexin V flow cytometry plots (FIG. 15C) of control LNA ASO (left) and LNA ASO1 (right) cells.

FIG. 16A is a graph of individual-nucleotide resolution cross-linking immunoprecipitation (iCLIP). RT-qPCR tiling primers spanning LINC01432 shows direct binding to CELF2 with Tiling Primers compared to IgG negative control in RPMI 8226 cells.

FIG. 16B is a graph of iCLIP showing U266B1 cells treated with high-dose melphalan for 48 hours. RT-qPCR tiling primers are compared to control cells.

FIG. 17 is a schematic of the pipeline to identify lncRNA in multiple myeloma poor responders (<24 months progression free survival) versus standard responders (>24 months progression free survival) to standard therapy (HDM and ASCT).

FIG. 18 is a schematic showing the identification and characterization of lncRNA in multiple myeloma poor responders and standard responders.

### DETAILED DESCRIPTION OF THE INVENTION

The present disclosure is based, at least in part, on the discovery of deregulated lncRNAs with poor response to high-dose melphalan (HDM) autologous stem cell transplant (ASCT) with a 3-drug regimen. As shown herein, long non-coding RNA LINCOOXPRR is upregulated in newly diagnosed multiple myeloma promoting poor response to high-dose melphalan (HDM) with autologous stem cell transplantation (ASCT) and three-drug regimen.

The present invention relates to compositions and methods for newly diagnosed cancer and metastatic disease diagnosis, research, and therapy, including but not limited to, cancer markers. Specifically, the present invention relates to the use of a long non-coding RNA as a prognostic or diagnostic marker and clinical target for multiple myeloma.

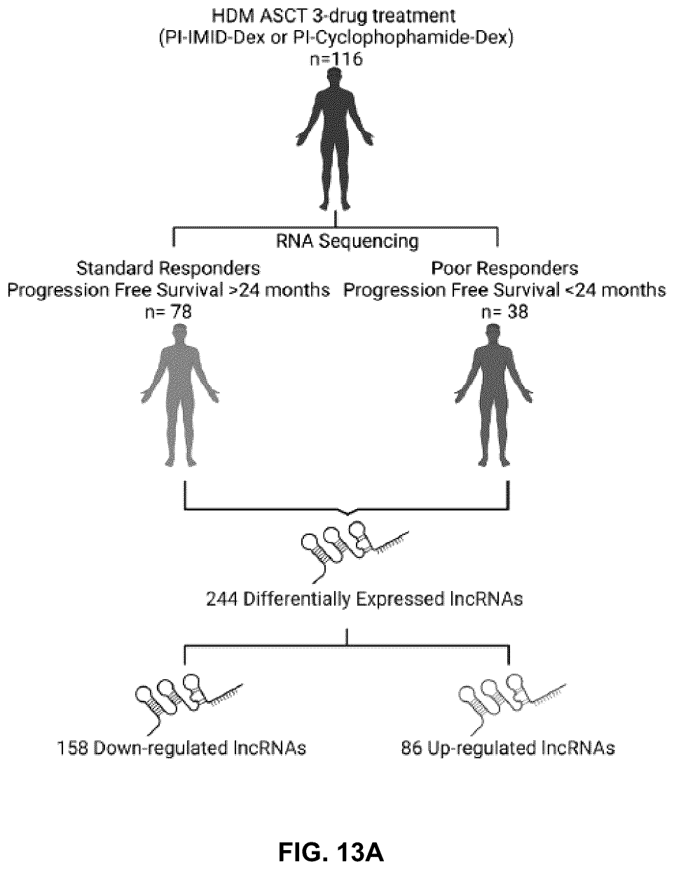

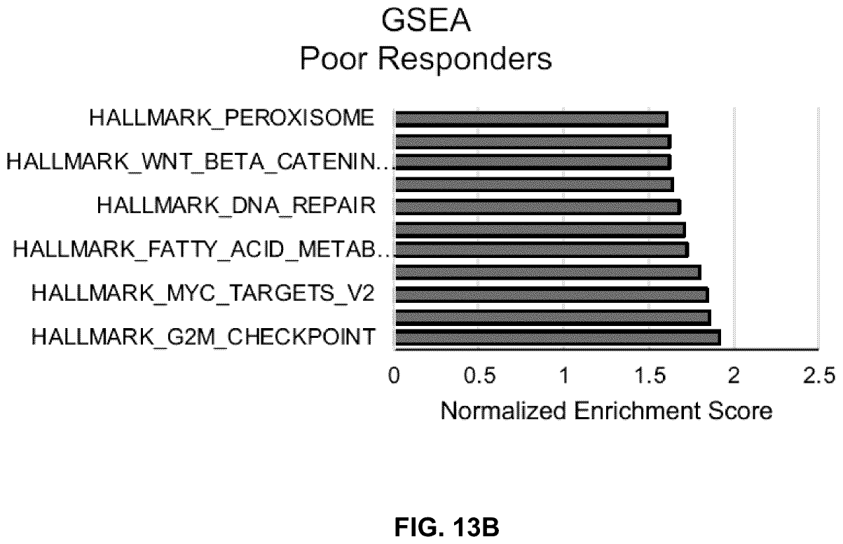

One aspect of the present disclosure provides for the identification of deregulated lncRNAs with poor response to high-dose melphalan (HDM) autologous stem cell transplant (ASCT) with a 3-drug regimen. In order to identify which lncRNAs are associated with poor response to the standard treatment of HDM with ASCT followed by induction with a 3-drug regimen consisting of a protease inhibitor-immunomodulatory drug-dexamethasone or protease inhibitor-cyclophosphamide-dexamethasone treatment, transcriptome sequencing data of 116 newly diagnosed multiple myeloma patient CD138 sorted bone marrow samples from the Multiple Myeloma Research Foundation (MMRF) Clinical Outcomes in Multiple Myeloma to Personal Assessment of Genetic Profiles (CoMMpass) study were analyzed. We grouped samples into two cohorts consisting of those who had progression-free survival >24 months termed ‘Standard Responders’ (n=78) and those with <24 months of progression-free survival termed ‘Poor Responders’ (n=38) after receiving therapy. 86 differentially expressed up-regulated lncRNAs and 158 down-regulated lncRNAs were identified when comparing Poor Responders to Standard Responders (FIG. 13A). Gene set enrichment analysis was next conducted using all differentially expressed RNAs from Poor Responders and discovered high enrichment of gene sets associated with G2M checkpoint, E2F, MYC, and also mitotic spindle and DNA repair pathways (FIG. 13B).

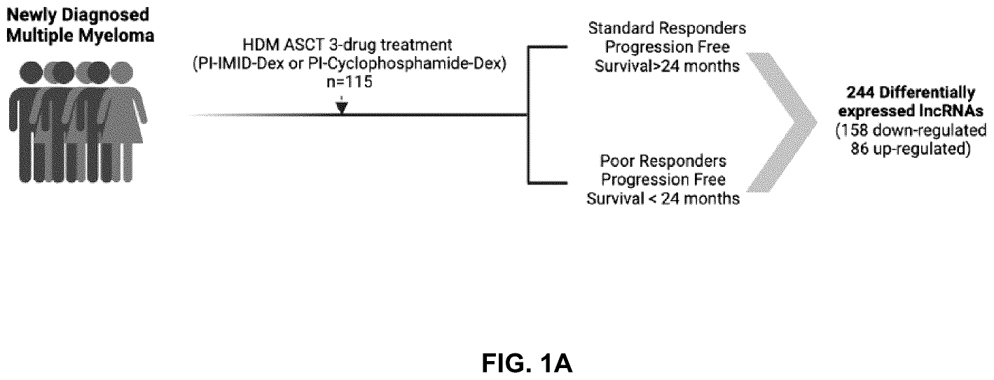

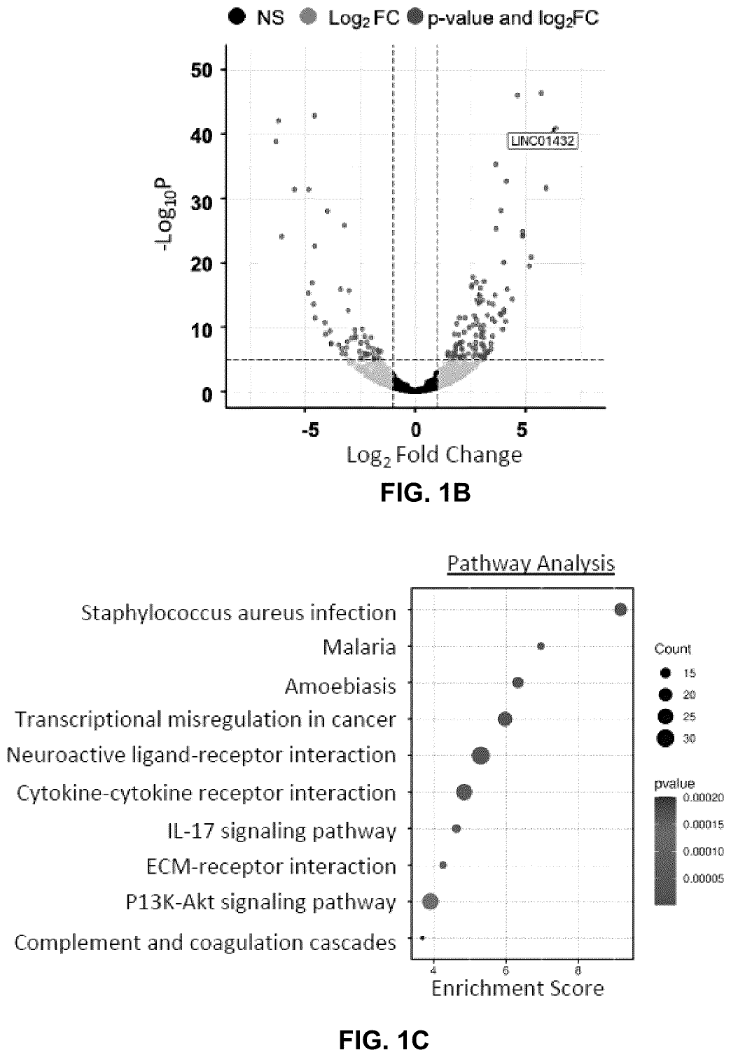

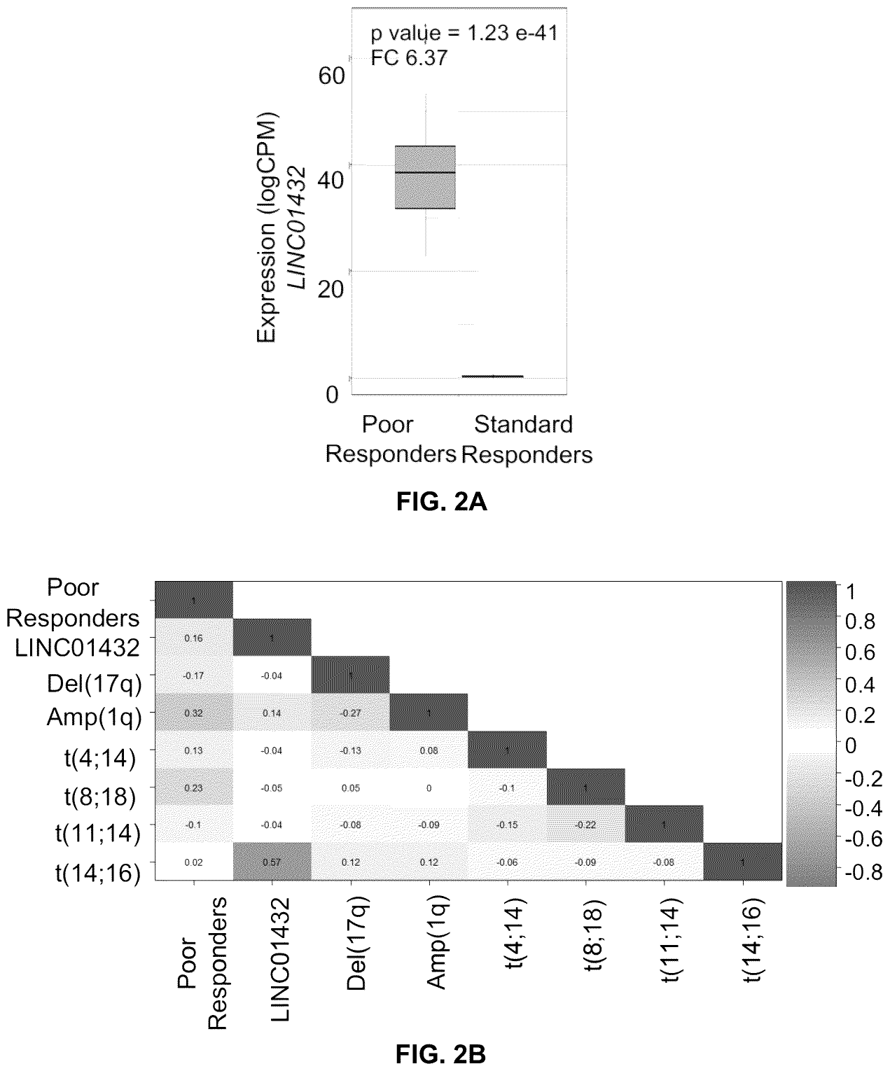

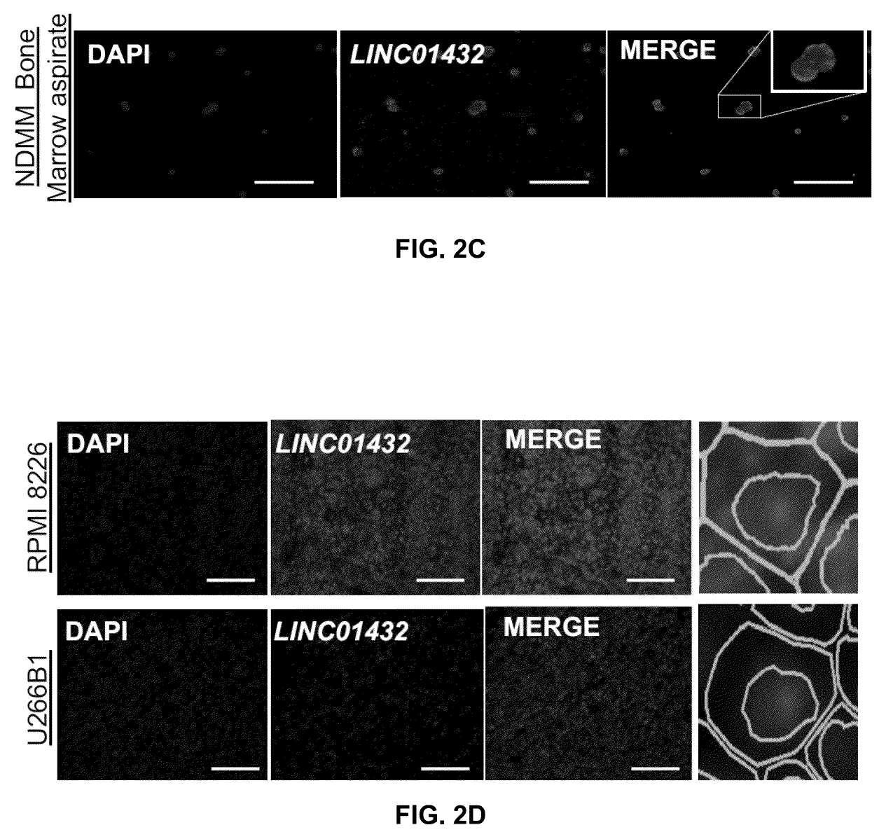

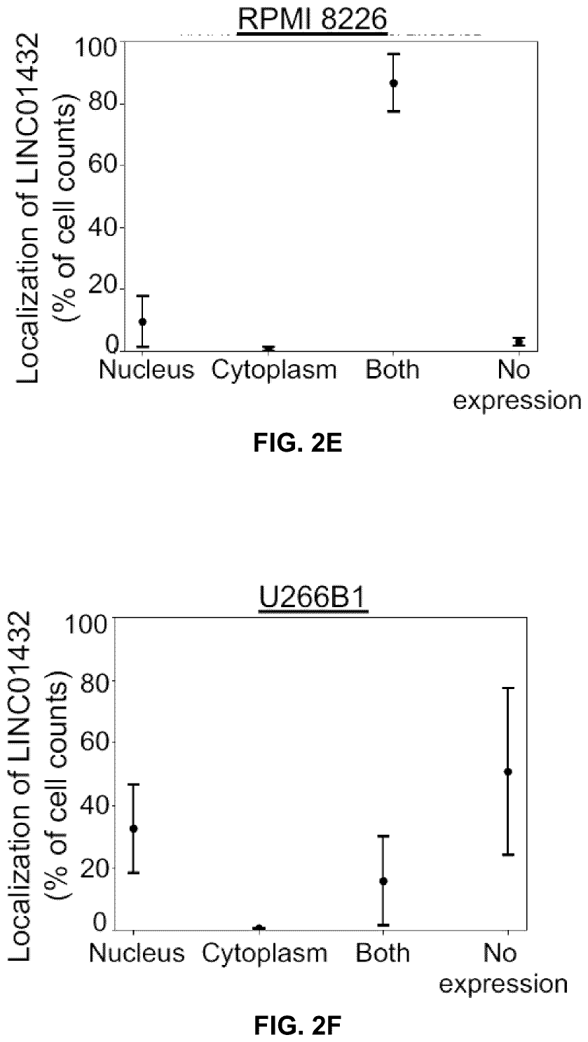

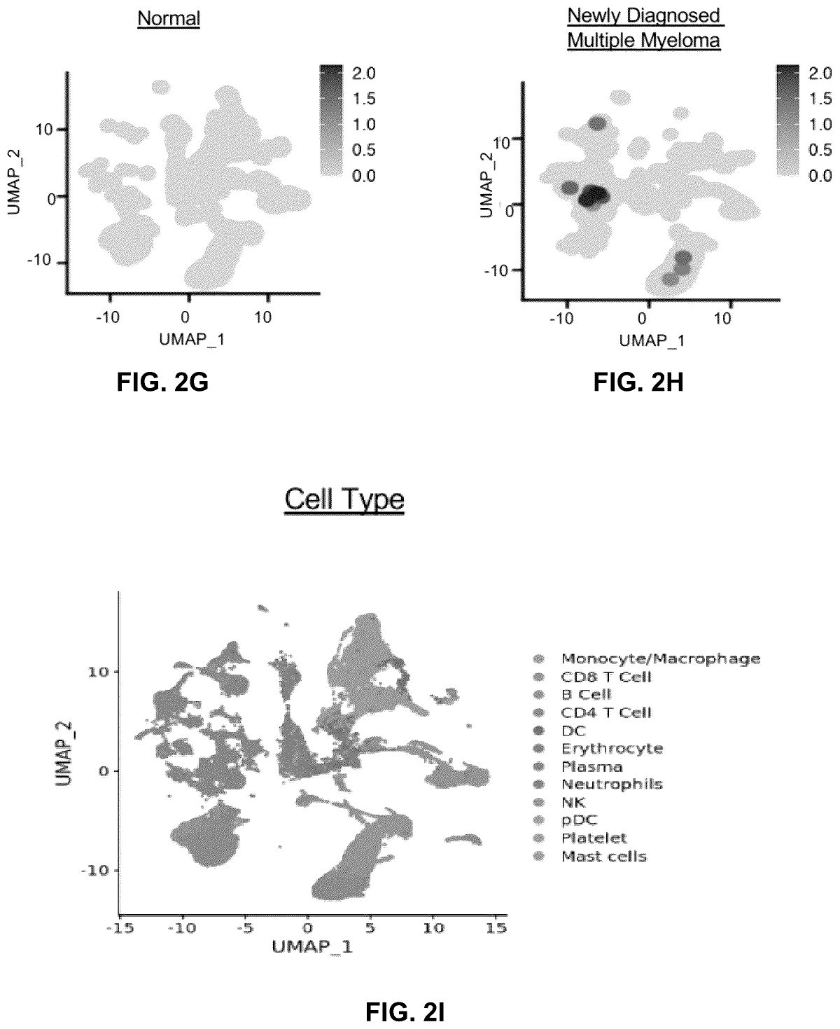

As described in the examples herein, efforts were focused on characterizing the lncRNA found to be most significantly up-regulated when comparing Poor Responders to Standard Responders (Fold change=6.37, p-value=1.23e-41, FIG. 1B, FIG. 1C). LINC01432 is a long intergenic non-protein coding RNA located on chromosome 20, has four exons, and is 693 nucleotides long. There is little to no knowledge about LINC01432 as it has only been associated with containing a SNP in a single-trait genome-wide association study for male baldness. LINC01432 was characterized by detecting genetic sub-types of myeloma, due to the heterogeneity and detection of hyperploidy in several chromosomes of myeloma patients. It was determined that LINC01432 had high correlation expression with samples that contained t(14; 16) and Amp (1q) translocations (FIG. 2B). Next, LINC01432 expression in single-cell RNA sequencing data was assessed, using eight normal bone marrow mononuclear cells taken from healthy donors and 13 newly diagnosed multiple myeloma bone marrow samples. High expression of LINC01432 was discovered in plasma cells from the newly diagnosed multiple myeloma samples with no expression in normal samples (FIG. 2G, FIG. 2H, FIG. 2I).

In some aspects, non-responders to Mephalan treatment may be characterized as having a LINC01432 expression level higher than a healthy control expression level. In some aspects, the threshold level comprises a LINC01432 expression level of about 6.2-6.7 fold change relative to a healthy control expression level. In one aspect, the threshold level comprises a LINC01432 expression level of 6.42 fold change relative to a healthy control expression level.

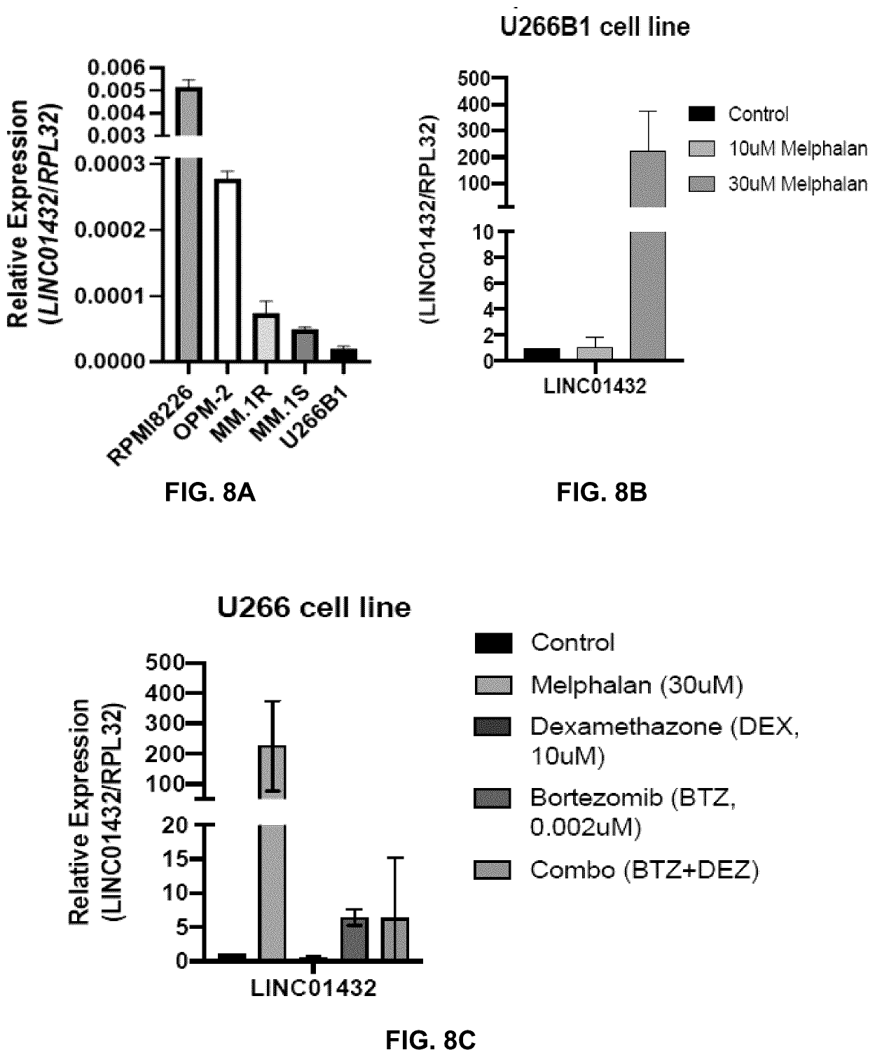

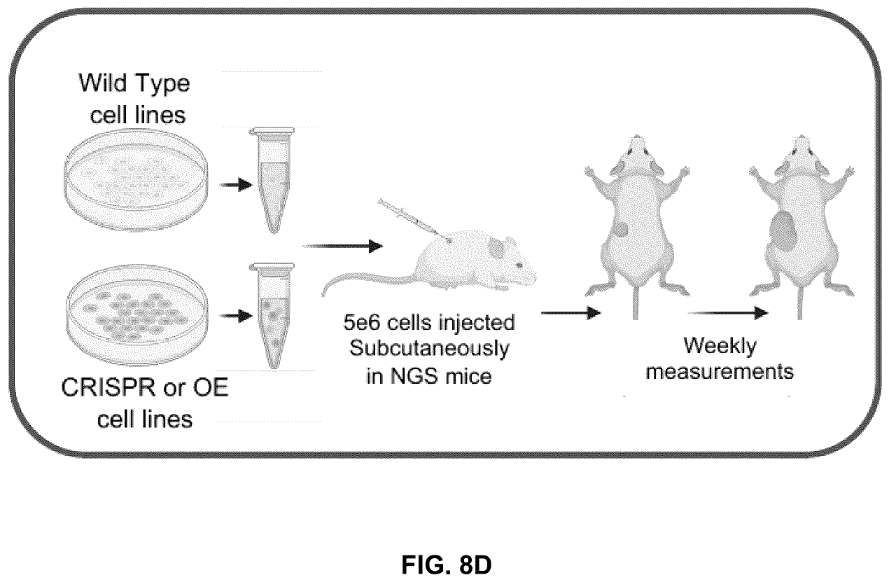

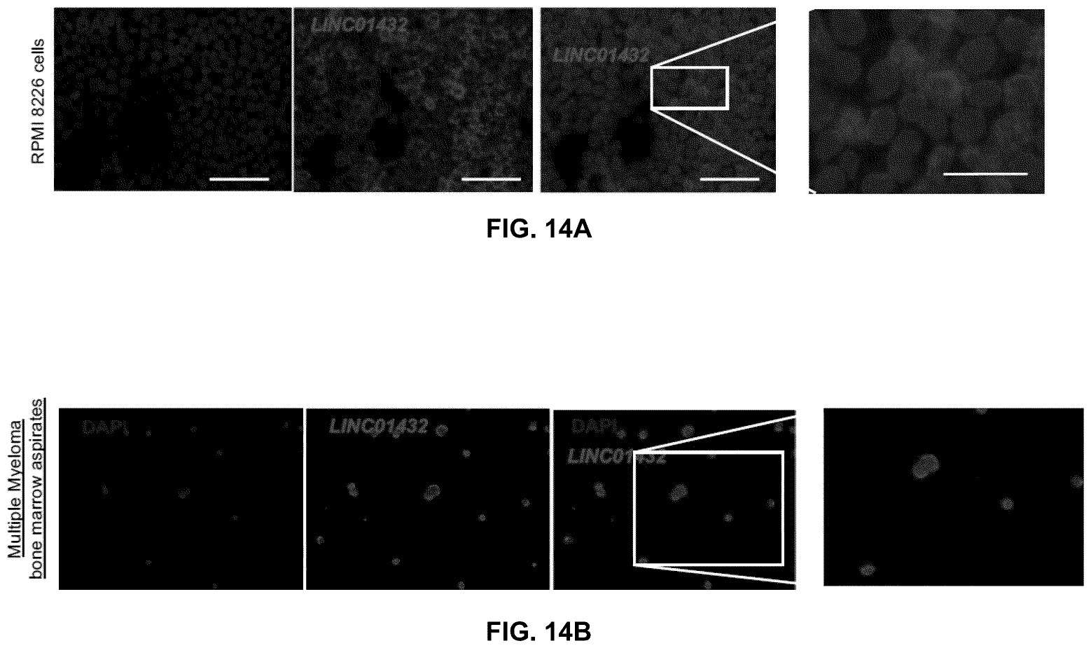

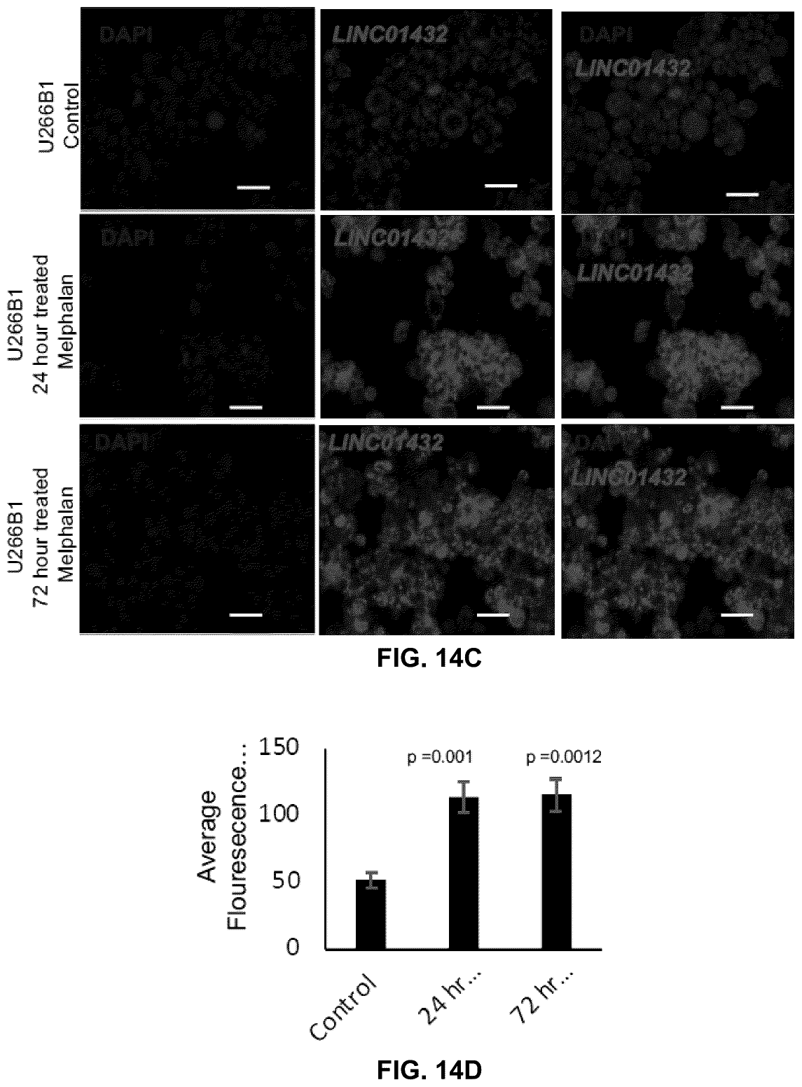

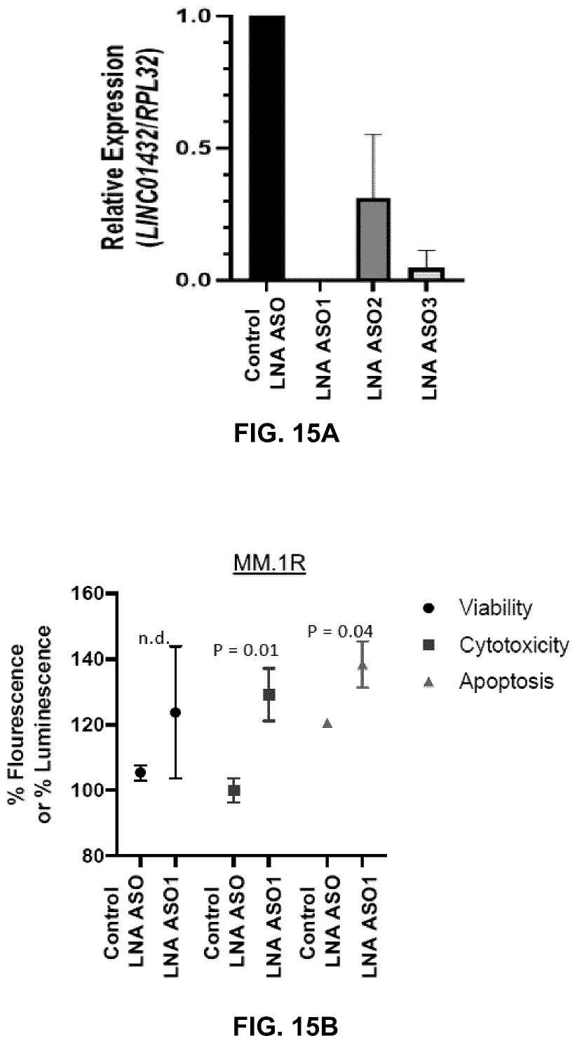

In another aspect, LINC01432 is expressed in myeloma cell lines and increases expression with Melphalan treatment. To characterize the functions of LINC01432, its expression was analyzed in a panel of multiple myeloma cell lines. LINC01432 is highly expressed in RPMI 8226 and OPM-2 cell lines and contains low expression in MM.1S and U266B1 cell lines (FIG. 8A). Next, a cell line with low LINC01432 expression, U266B1, was treated with standard drugs used in patients to mimic our sequencing cohort, including Melphalan, Dexamethasone, Bortezomib, or combinations. High-dose melphalan (30 mM) increases LINC01432 expression (FIG. 8B and FIG. 8C). High expression of LINC01432 in RPMI 8226 cells (FIG. 14A) and U266B1 cells treated with high-dose melphalan for 24 hours or 48 hours (FIG. 14C, 24 hours p=0.001, 48 hours p=0.0012)) was further validated using multiplexed Fluorescent RNA In situ Hybridization (mFISH). Lastly, LINC01432 expression was detected in unsorted multiple myeloma patient bone marrow aspirates (FIG. 14B).

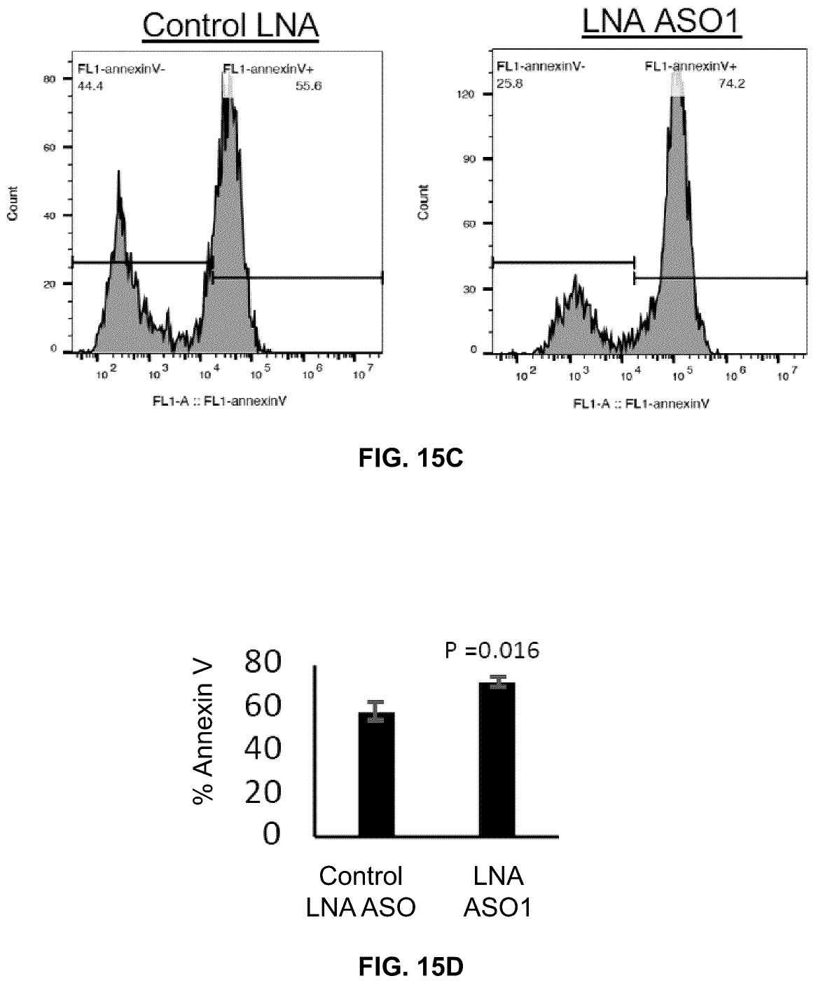

In yet another aspect, LINC01432 can increase apoptosis. In order to gain a better understanding of the role LINC01432 plays in poor response to current therapies and to test its therapeutic potential, in vitro-locked nucleic acid GapmeR antisense oligonucleotides (LNA ASOs), which are increasingly being used in clinical trials, were used to silence the expression of LINC01432 in RPMI 8226 and MM.1R cell lines. Utilizing three custom-designed LINC01432 LNA ASOs (FIG. 15A), we are able to achieve a sufficient decrease in LINC001432 expression (FIG. 15A). MM.1R cells were next treated with LINC01432 LNA ASO1 and a significant increase in cytotoxicity (p=0.01) and apoptosis (p=0.04) was detected using ApoTox-Glo Triplex Assay, suggesting the therapeutic potential of LINC01432. GFP-luciferase overexpression constructs containing full-length LINC01432 were next created, and LINC01432 was transiently overexpressed in U266B1 cells to detect an increase in viability (p=0.001) and a decrease in apoptosis (p=0.04) as compared to empty vector control (FIG. 15C, FIG. 15D). These experiments provide evidence that LINC01432 is highly expressed in myeloma cell lines and is associated with inhibiting apoptosis.

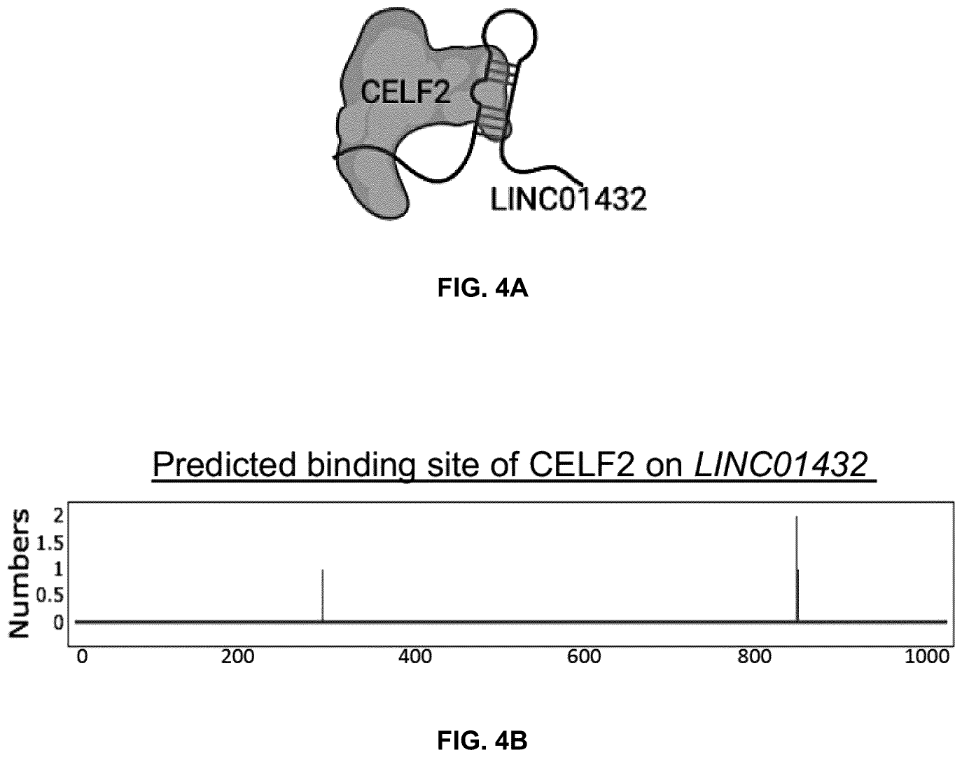

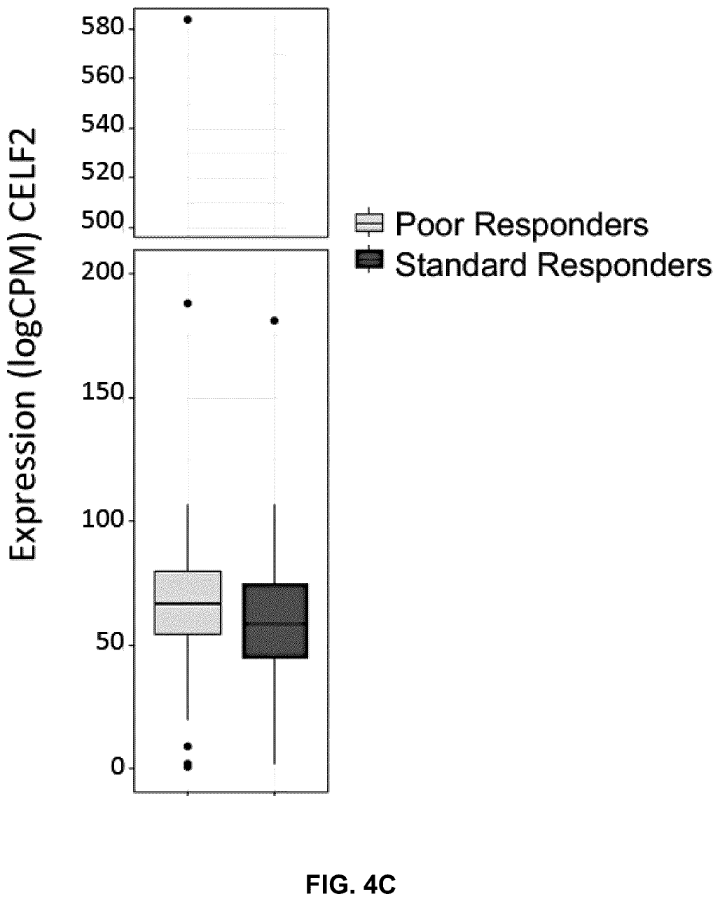

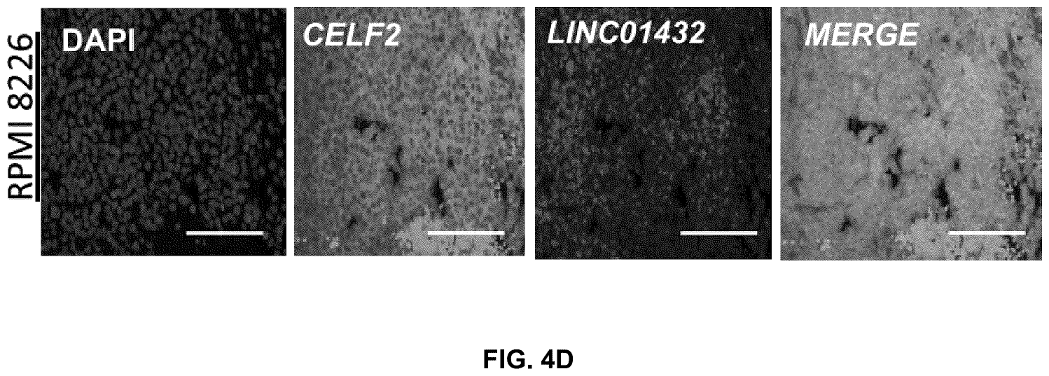

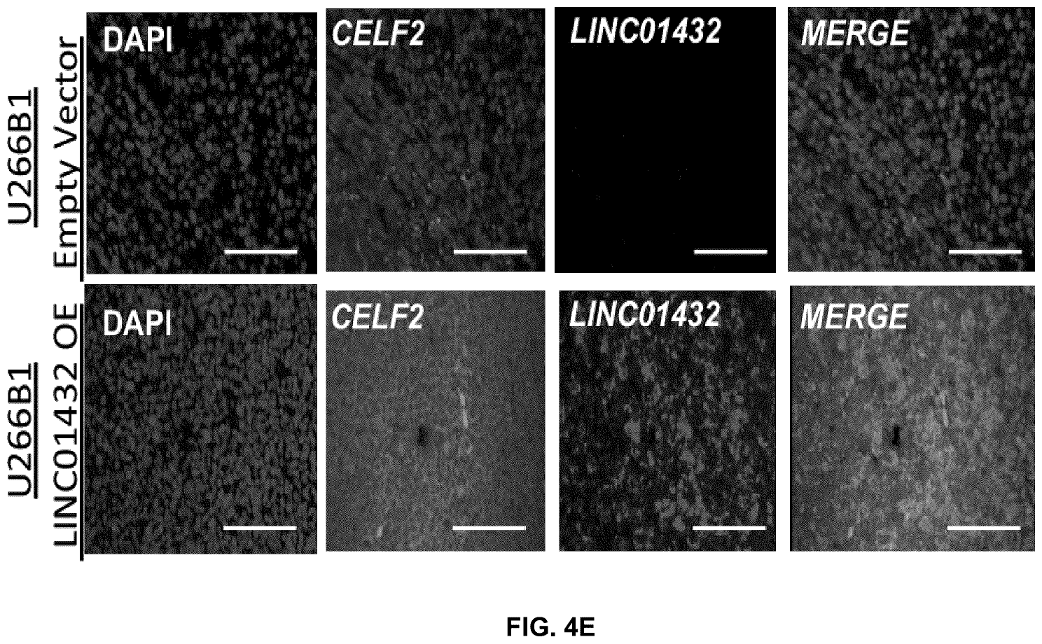

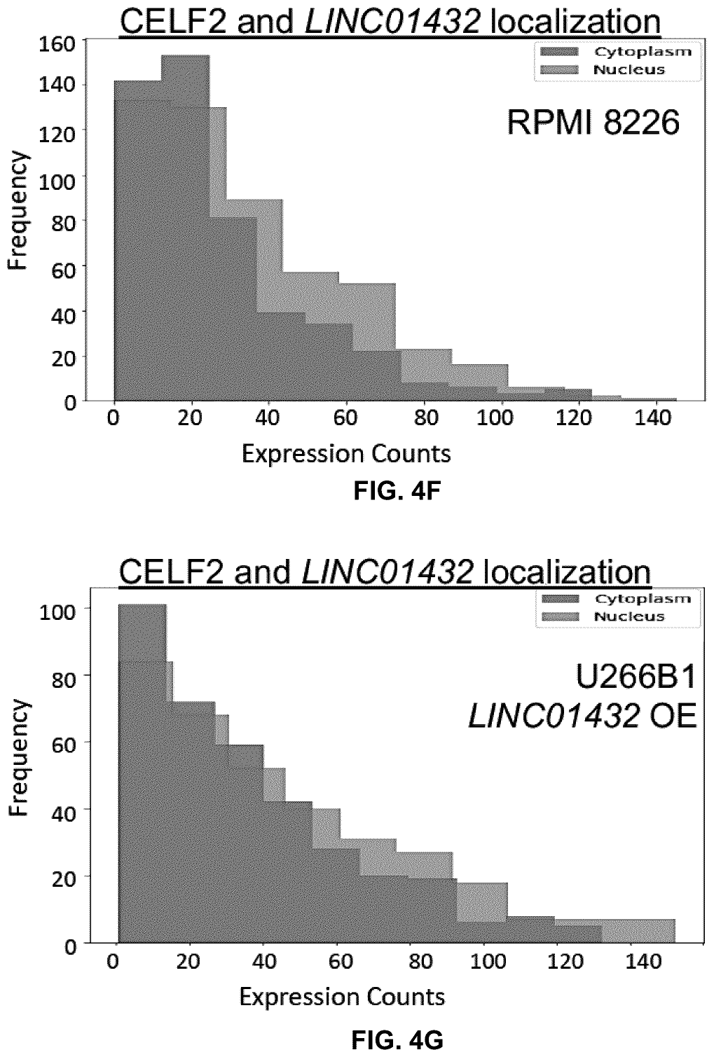

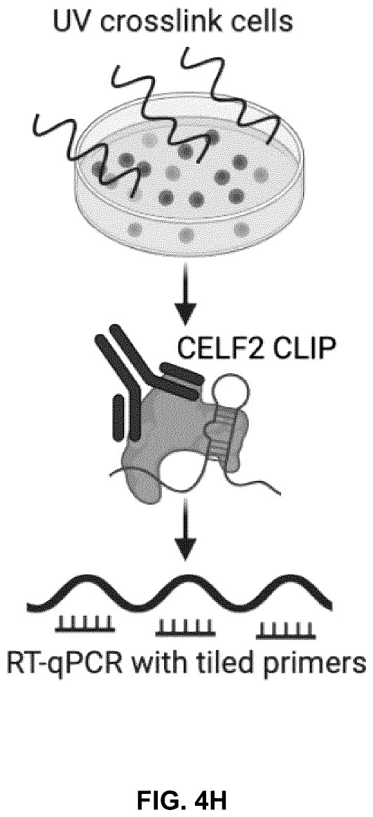

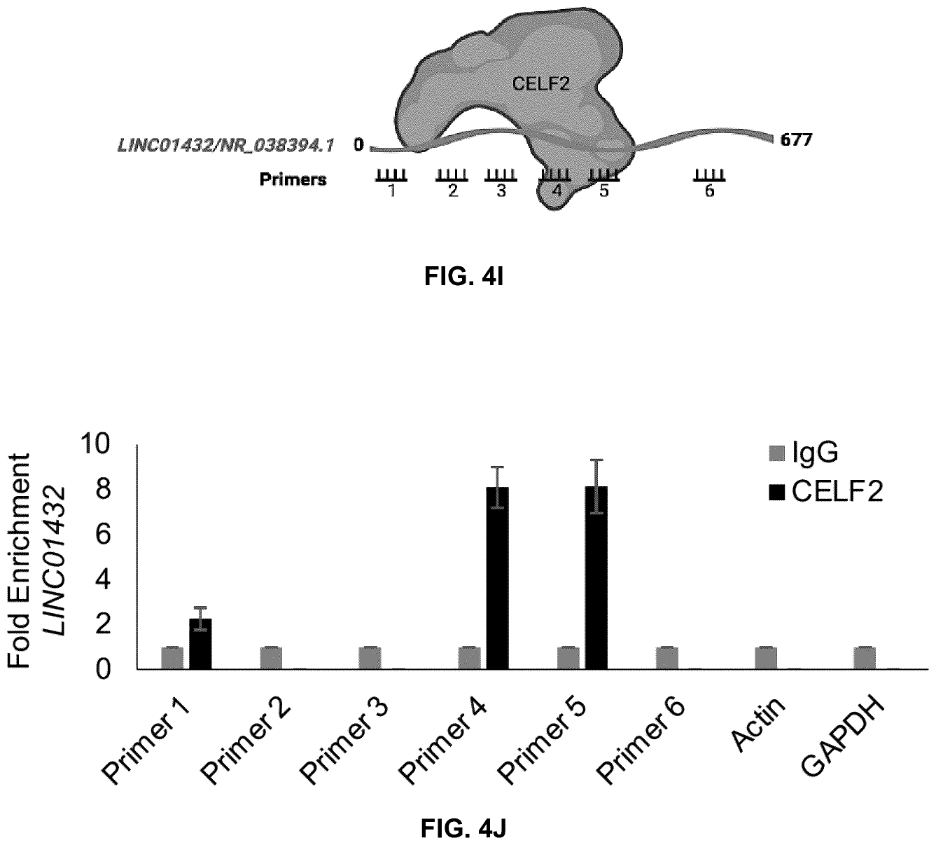

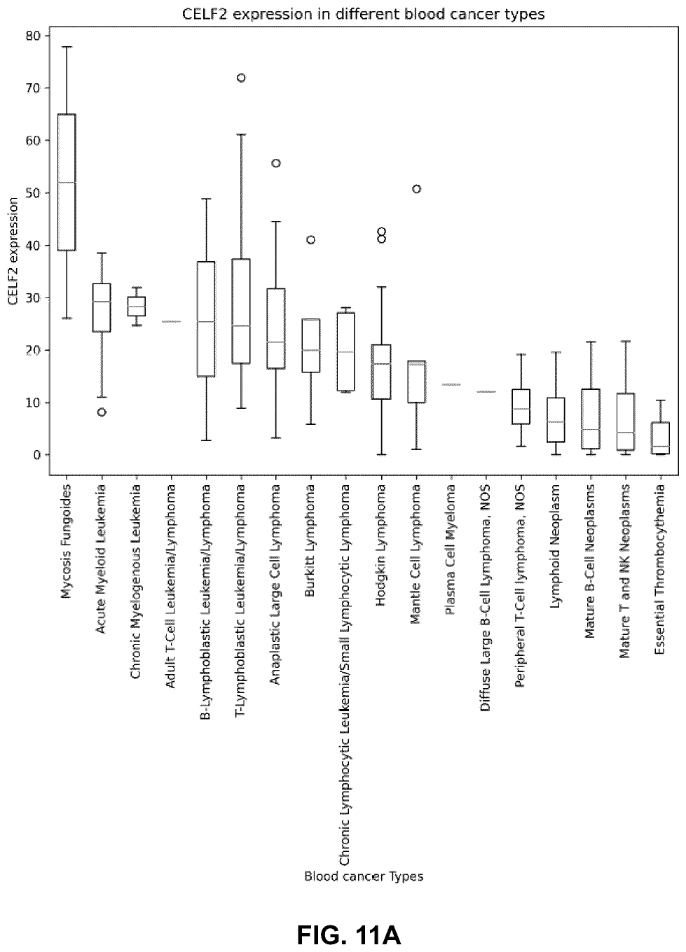

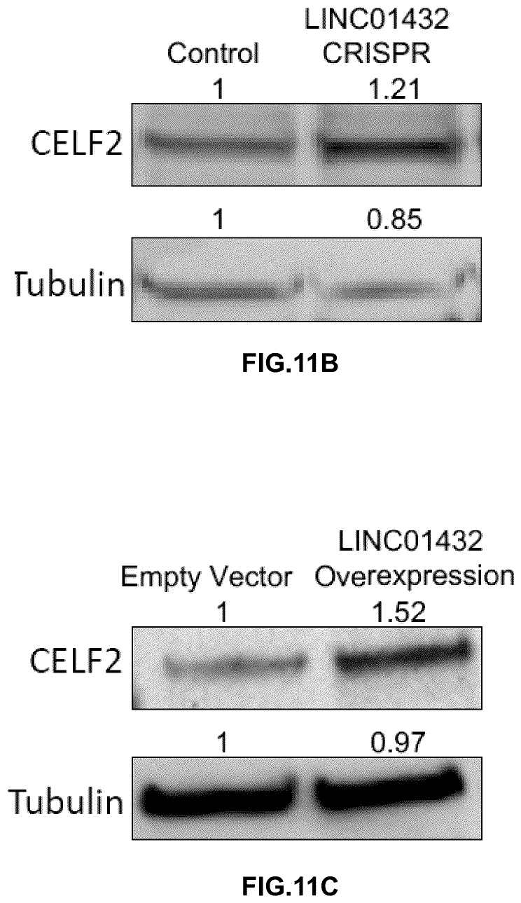

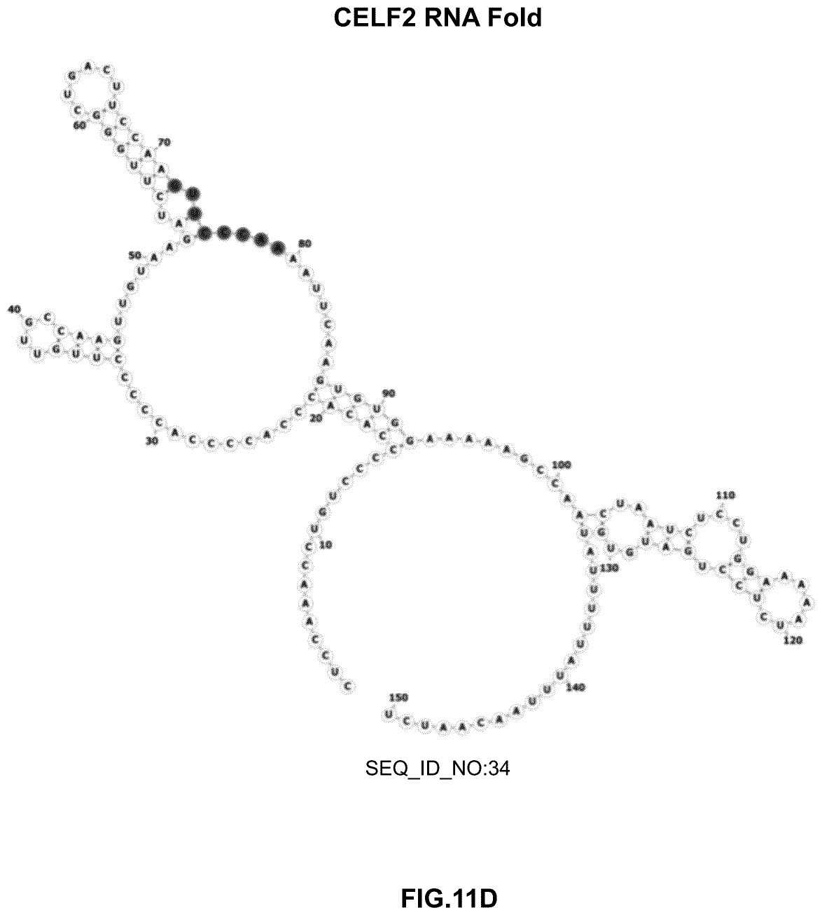

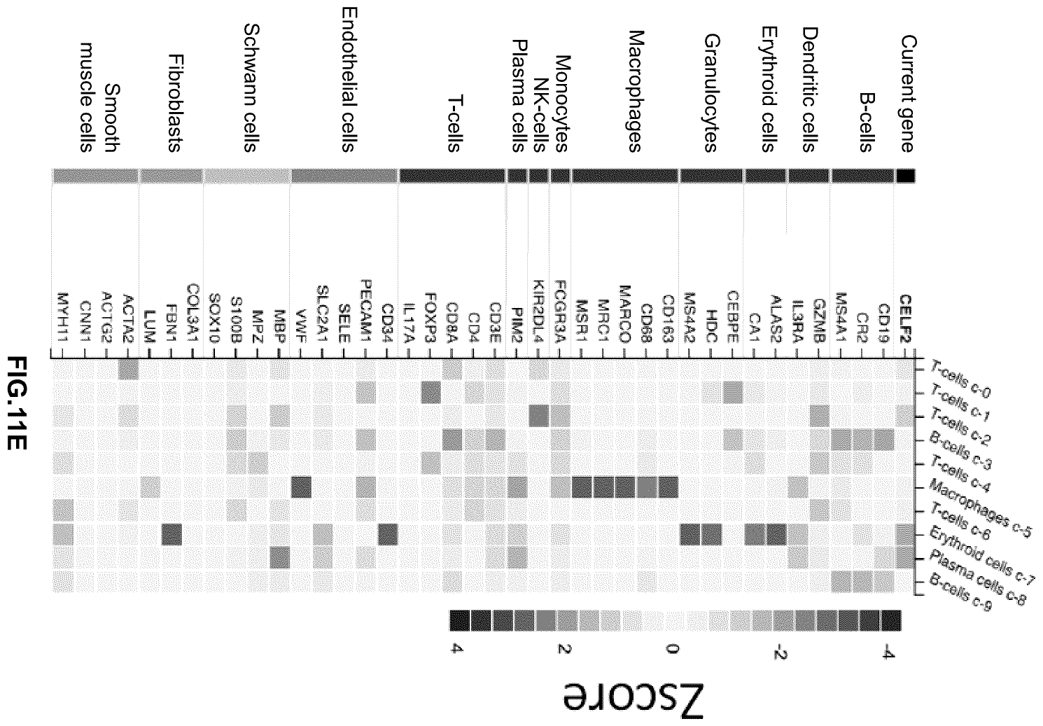

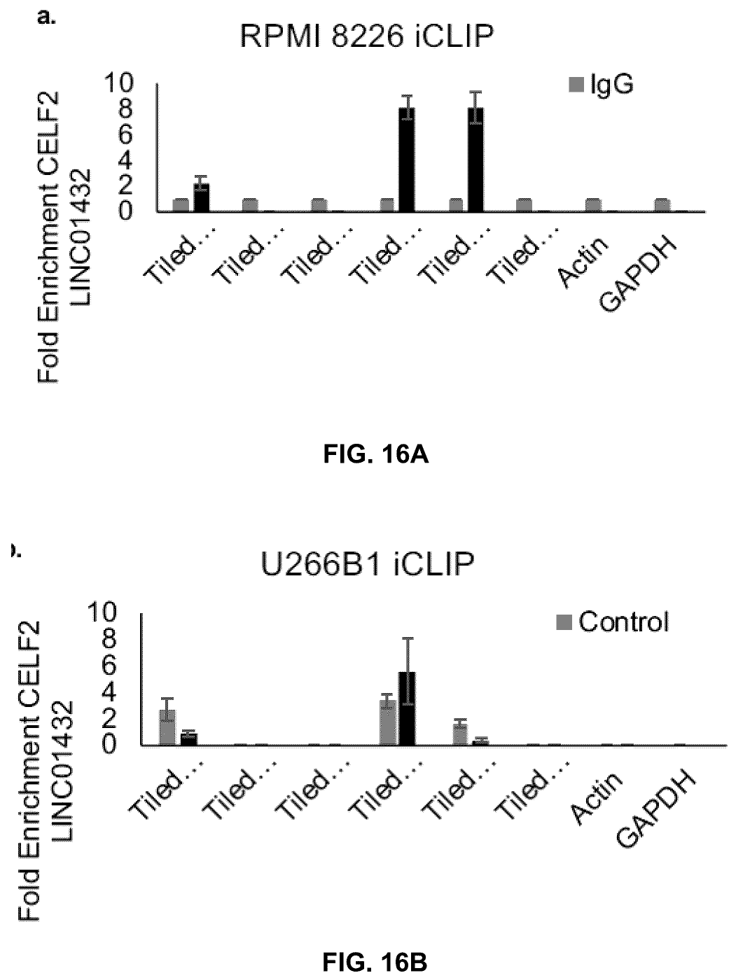

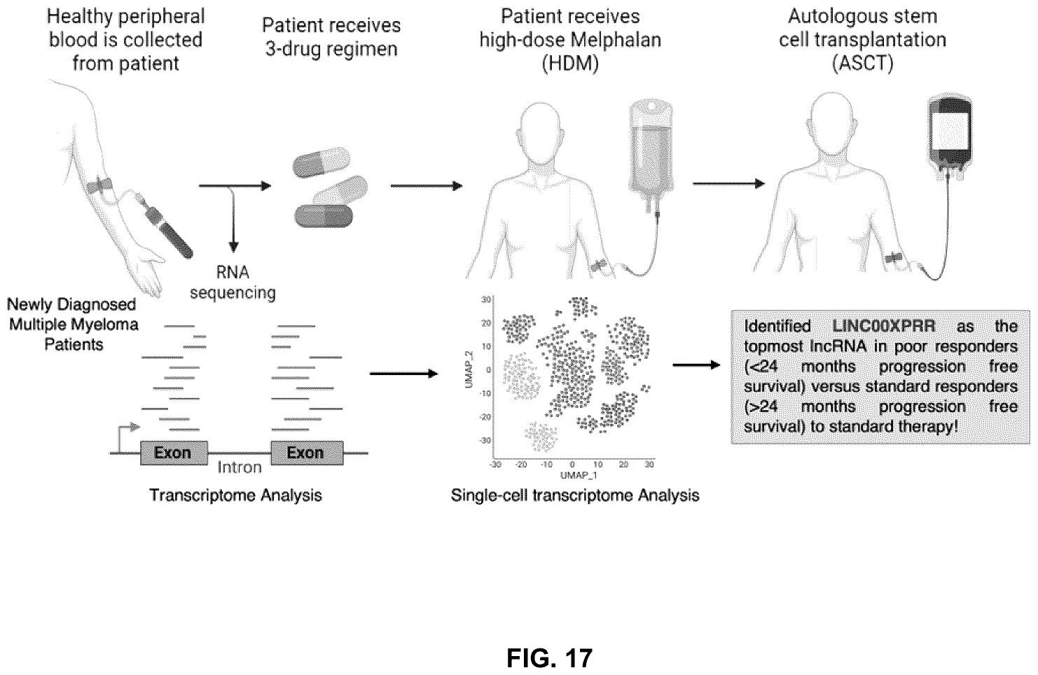

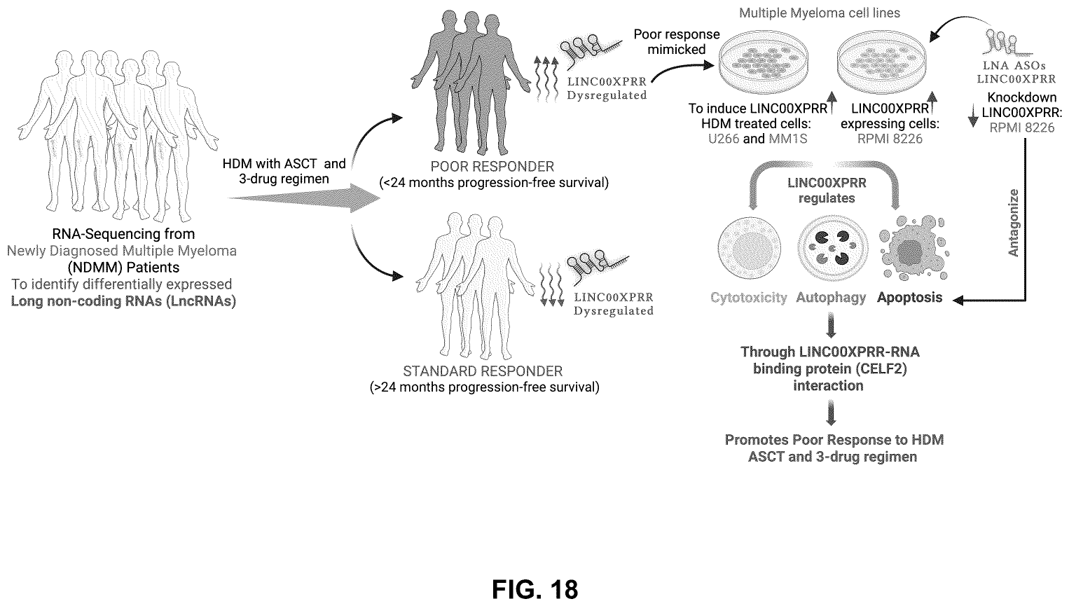

In accordance with another aspect, LINC01432 binds to CELF2 protein. Many studies on determining lncRNA function have found that lncRNAs play important roles in cancer and therapy resistance due to their capability to bind with proteins and regulate downstream genes. As there is limited information on the functions of LINC01432, POSTAR361 was utilized as a first step to identify any proteins that may bind to LINC01432. POSTAR3 is a unique comprehensive database for exploring post-transcriptional regulation and RNA-binding proteins that incorporate publicly available large-scale CLIP-sequencing, Ribo-sequencing datasets, RNA secondary structure, and also miRNA-mediated degradation events. Interestingly, there were only two RNA-binding proteins, CELF2 and AGO2, found to bind to LINC01432 in publicly available CLIP-sequencing datasets (FIG. 10). Although the determined score of binding was low (0.019) for both proteins, CELF2 CLIP-sequencing was done using human JSL1 T cells, and assessing CELF2 expression in blood cell types showed high expression in plasma cells (FIG. 11D, FIG. 11E), thus its significance in myeloma was determined. CELF (CUGBP Elav-like family) proteins are RNA-binding proteins with pleiotropic capabilities in RNA processing that have been found to compete with non-coding RNAs including lncRNAs. CELF2 has previously been found to bind lncRNAs to promote proliferation, migration, and tumor growth of multiple cancers by regulating downstream mRNAs, however, has not yet been studied in myeloma. High expression of CELF2 was found in the RNA sequencing data (log CPM >50), but a significant difference in expression was not observed when comparing Poor Responders to Standard Responders (FIG. 4C). Next, to identify the regions of LINC01432 that may directly bind to CELF2, individual-nucleotide resolution cross-linking immunoprecipitation (iCLIP), which uses UV cross-linking with immunoprecipitation to precisely map RNA binding protein binding sites, coupled with RT-qPCR, was conducted. RT-qPCR tiling primers spanning LINC01432 shows direct binding to CELF2 with Tiling Primer 1 (Fold Change >2), Tiling Primer 4 (Fold Change >8), and Tiling Primer 5 (Fold Change >8) compared to IgG negative control in RPMI 8226 cells (FIG. 16A). U266B1 cells were next treated with high-dose melphalan for 48 hours to detect a decrease in binding enrichment compared to control cells with Tiling Primer 1 and Tiling Primer 5 (Fold Change >2, FIG. 16B). These results indicate that LINC01432 binds to CELF2 protein in myeloma and is not bound in the melphalan setting.

### Long Non-Coding RNA (LncRNA) Modulation Agents

As described herein, lncRNA expression has been implicated in various diseases, disorders, and conditions. As such, modulation of lncRNA (e.g., modulation of LINC01432) can be used for the treatment of such conditions. A lncRNA modulation agent can modulate lncRNA response or induce or inhibit lncRNA. LncRNA modulation can comprise modulating the expression of lncRNA in cells, modulating the quantity of cells that express lncRNA, or modulating the quality of the lncRNA cells.

LncRNA modulation agents can be any composition or method that can modulate lncRNA expression on cells (e.g., LINC01432). For example, a lncRNA modulation agent can be an activator, an inhibitor, an agonist, or an antagonist. As another example, the lncRNA modulation can be the result of gene editing.

An lncRNA modulation agent can be an anti-lncRNA antibody (e.g., a monoclonal antibody to LINC01432).

An lncRNA modulating agent can be an agent that induces or inhibits progenitor cell differentiation into lncRNA-expressing cells (e.g., a deregulated lncRNA such as LINC01432). For example, antisense oligonucleotides (LNA ASOs) can be used to block lncRNAs such as LINC01432.

Long Non-Coding RNA (lncRNA) Signal Reduction, Elimination, or Inhibition by Small Molecule Inhibitors, shRNA, siRNA, or ASOs

As described herein, an lncRNA modulation agent can be used for use in multiple myeloma therapy. An lncRNA modulation agent can be used to reduce/eliminate or enhance/increase lncRNA signals. For example, an lncRNA modulation agent can be a small molecule inhibitor of an lncRNA such as LINC01432. As another example, an lncRNA modulation agent can be a short hairpin RNA (shRNA). As another example, an lncRNA modulation agent can be a short interfering RNA (siRNA).

As another example, RNA (e.g., long noncoding RNA (lncRNA)) can be targeted with antisense oligonucleotides (ASOs) as a therapeutic. Processes for making ASOs targeted to RNAs are well known; see e.g. Zhou et al. 2016 Methods Mol Biol. 1402:199-213. Except as otherwise noted herein, therefore, the process of the present disclosure can be carried out in accordance with such processes.

### Long Non-Coding RNA (LncRNA) Inhibiting Agent

One aspect of the present disclosure provides for the targeting of an lncRNA including but not limited to LINC01432, its receptor, or its downstream signaling. The present disclosure provides methods of treating or preventing multiple myeloma based on the discovery of deregulated lncRNAs with poor response to high dose melphalan (HDM) autologous stem cell transplant (ASCT) with 3-drug regimen.

As described herein, inhibitors of lncRNA (e.g., antibodies, fusion proteins, small molecules) can reduce or prevent multiple myeloma. An lncRNA inhibiting agent can be any agent that can inhibit an lncRNA, downregulate an lncRNA, or knockdown an lncRNA, including but not limited to LINC01432.

As an example, an lncRNA inhibiting agent can inhibit LINC01432 signaling.

For example, the lncRNA inhibiting agent can be an anti-lncRNA antibody. As an example, the anti-lncRNA antibody can be an anti-LINC01432 antibody, an anti-CELF2 antibody, or an anti-lncRNA antibody with activity against any combination of LINC01432 and CELF2. Furthermore, the anti-lncRNA antibody can be a murine antibody, a humanized murine antibody, or a human antibody.

As another example, the lncRNA inhibiting agent can be an anti-LINC01432 antibody, wherein the anti-LINC01432 antibody prevents binding of LINC01432 to its receptor or prevents activation of LINC01432 and downstream signaling.

As another example, the lncRNA inhibiting agent can be a fusion protein. For example, the fusion protein can be a decoy receptor for LINC01432. Furthermore, the fusion protein can comprise a mouse or human Fc antibody domain fused to the ectodomain of LINC01432.

As another example, an lncRNA inhibiting agent can be in vitro-locked nucleic acid GapmeR antisense oligonucleotides (LNA ASOs), which have been shown to be potent and specific inhibitors of lncRNA (including LINC01432 signaling.

As another example, an lncRNA inhibiting agent can be an inhibitory protein that antagonizes LINC01432. For example, the lncRNA inhibiting agent can be a viral protein, which has been shown to antagonize LINC01432.

As another example, an lncRNA inhibiting agent can be a short hairpin RNA (shRNA) or a short interfering RNA (siRNA) targeting LINC01432 or CELF2.

As another example, an lncRNA inhibiting agent can be an sgRNA targeting LINC01432 or CELF2.

Methods for preparing an lncRNA inhibiting agent (e.g., an agent capable of inhibiting LINC01432 signaling) can comprise the construction of a protein/Ab scaffold containing the natural LINC01432 receptor as an LINC01432 neutralizing agent; developing inhibitors of the LINC01432 receptor “down-stream”; or developing inhibitors of the LINC01432 production “up-stream”.

Inhibiting a lncRNA can be performed by genetically modifying a lncRNA in a subject or genetically modifying a subject to reduce or prevent expression of the lncRNA gene, such as through the use of CRISPR-Cas9 or analogous technologies, wherein, such modification reduces or prevents multiple myeloma.

### Chemical Agent:

Examples of lncRNA modulation agents are described herein.

R groups can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; and aryl comprising a phenyl; heteroaryl containing from 1 to 4 N, O, or S atoms; unsubstituted phenyl ring; substituted phenyl ring; unsubstituted heterocyclyl; and substituted heterocyclyl, wherein the unsubstituted phenyl ring or substituted phenyl ring can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; straight chain or branched C₁₋₁₀alkyl amine; heterocyclyl; heterocyclic amine; aryl comprising a phenyl; and heteroaryl containing from 1 to 4 N, O, or S atoms; and the unsubstituted heterocyclyl or substituted heterocyclyl can be optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₁₀cycloalkyl optionally containing unsaturation or one oxygen or nitrogen atom; heterocyclyl; straight chain or branched C₁₋₁₀alkyl amine; heterocyclic amine; and aryl comprising a phenyl; and heteroaryl containing from 1 to 4 N, O, or S atoms. Any of the above can be further optionally substituted.

The term “imine” or “imino”, as used herein, unless otherwise indicated, can include a functional group or chemical compound containing a carbon-nitrogen double bond. The expression “imino compound”, as used herein, unless otherwise indicated, refers to a compound that includes an “imine” or an “imino” group as defined herein. The “imine” or “imino” group can be optionally substituted.

The term “hydroxyl”, as used herein, unless otherwise indicated, can include —OH. The “hydroxyl” can be optionally substituted.

The terms “halogen” and “halo”, as used herein, unless otherwise indicated, include chlorine, chloro, Cl; fluorine, fluoro, F; bromine, bromo, Br; or iodine, iodo, or I.

The term “acetamide”, as used herein, is an organic compound with the formula CH₃CONH₂. The “acetamide” can be optionally substituted.

The term “aryl”, as used herein, unless otherwise indicated, includes a carbocyclic aromatic group. Examples of aryl groups include, but are not limited to, phenyl, benzyl, naphthyl, or anthracenyl. The “aryl” can be optionally substituted.

The terms “amine” and “amino”, as used herein, unless otherwise indicated, include a functional group that contains a nitrogen atom with a lone pair of electrons and wherein one or more hydrogen atoms have been replaced by a substituent such as, but not limited to, an alkyl group or an aryl group. The “amine” or “amino” group can be optionally substituted.

The term “alkyl”, as used herein, unless otherwise indicated, can include saturated monovalent hydrocarbon radicals having straight or branched moieties, such as but not limited to, methyl, ethyl, propyl, butyl, pentyl, hexyl, octyl groups, etc. Representative straight-chain lower alkyl groups include, but are not limited to, -methyl, -ethyl, -n-propyl, -n-butyl, -n-pentyl, -n-hexyl, -n-heptyl and -n-octyl; while branched lower alkyl groups include, but are not limited to, -isopropyl, -sec-butyl, -isobutyl, -tert-butyl, -isopentyl, 2-methylbutyl, 2-methylpentyl, 3-methylpentyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methylhexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, unsaturated C₁₋₁₀ alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl, -acetylenyl, -propynyl, -1-butynyl, -2-butynyl, -1-pentynyl, -2-pentynyl, or -3-methyl-1 butynyl. An alkyl can be saturated, partially saturated, or unsaturated. The “alkyl” can be optionally substituted.

The term “carboxyl”, as used herein, unless otherwise indicated, can include a functional group consisting of a carbon atom double bonded to an oxygen atom and single bonded to a hydroxyl group (—COOH). The “carboxyl” can be optionally substituted.

The term “alkenyl”, as used herein, unless otherwise indicated, can include alkyl moieties having at least one carbon-carbon double bond wherein alkyl is as defined above and including E and Z isomers of said alkenyl moiety. An alkenyl can be partially saturated or unsaturated. The “alkenyl” can be optionally substituted.

The term “alkynyl”, as used herein, unless otherwise indicated, can include alkyl moieties having at least one carbon-carbon triple bond wherein alkyl is as defined above. An alkynyl can be partially saturated or unsaturated. The “alkynyl” can be optionally substituted.

The term “acyl”, as used herein, unless otherwise indicated, can include a functional group derived from an aliphatic carboxylic acid, by removal of the hydroxyl (—OH) group. The “acyl” can be optionally substituted.

The term “alkoxyl”, as used herein, unless otherwise indicated, can include O-alkyl groups wherein alkyl is as defined above, and O represents oxygen. Representative alkoxyl groups include, but are not limited to, —O-methyl, —O-ethyl, —O-n-propyl, —O-n-butyl, —O-n-pentyl, —O-n-hexyl, —O-n-heptyl, —O-n-octyl, —O— isopropyl, —O-sec-butyl, —O-isobutyl, —O-tert-butyl, —O-isopentyl, —O-2-methylbutyl, —O-2-methylpentyl, —O-3-methylpentyl, —O-2,2-dimethylbutyl, —O-2,3-dimethylbutyl, —O-2,2-dimethylpentyl, —O-2,3-dimethylpentyl, —O-3,3-dimethylpentyl, —O-2,3,4-trimethylpentyl, —O-3-methylhexyl, —O-2,2-dimethylhexyl, —O-2,4-dimethylhexyl, —O-2,5-dimethylhexyl, —O-3,5-dimethylhexyl, —O-2,4dimethylpentyl, —O-2-methylheptyl, —O-3-methylheptyl, —O-vinyl, —O-allyl, —O-1-butenyl, —O-2-butenyl, —O— isobutylenyl, —O-1-pentenyl, —O-2-pentenyl, —O-3-methyl-1-butenyl, —O-2-methyl-2-butenyl, —O-2,3-dimethyl-2-butenyl, —O-1-hexyl, —O-2-hexyl, —O-3-hexyl, —O— acetylenyl, —O-propynyl, —O-1-butynyl, —O-2-butynyl, —O-1-pentynyl, —O-2-pentynyl and —O-3-methyl-1-butynyl, —O-cyclopropyl, —O-cyclobutyl, —O-cyclopentyl, —O— cyclohexyl, —O-cycloheptyl, —O-cyclooctyl, —O-cyclononyl and —O-cyclodecyl, —O—CH2-cyclopropyl, —O—CH2-cyclobutyl, —O—CH2-cyclopentyl, —O—CH2-cyclohexyl, —O—CH2-cycloheptyl, —O—CH2-cyclooctyl, —O— CH2-cyclononyl, —O—CH2-cyclodecyl, —O—(CH2)2-cyclopropyl, —O—(CH2)2-cyclobutyl, —O—(CH2)2-cyclopentyl, —O—(CH2)2-cyclohexyl, —O—(CH2)2-cycloheptyl, —O—(CH2)2-cyclooctyl, —O—(CH2)2-cyclononyl, or —O—(CH2)2-cyclodecyl. An alkoxyl can be saturated, partially saturated, or unsaturated. The “alkoxyl” can be optionally substituted.

The term “cycloalkyl”, as used herein, unless otherwise indicated, can include an aromatic, non-aromatic, saturated, partially saturated, or unsaturated, monocyclic or fused, spiro or unfused bicyclic or tricyclic hydrocarbon referred to herein containing a total of from 1 to 10 carbon atoms (e.g., 1 or 2 carbon atoms if there are other heteroatoms in the ring), preferably 3 to 8 ring carbon atoms. Examples of cycloalkyls include, but are not limited to, C3-10 cycloalkyl groups include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl. The term “cycloalkyl” also can include -lower alkyl-cycloalkyl, wherein lower alkyl and cycloalkyl are as defined herein. Examples of -lower alkyl-cycloalkyl groups include, but are not limited to, —CH2-cyclopropyl, —CH2-cyclobutyl, —CH2-cyclopentyl, —CH2-cyclopentadienyl, —CH2-cyclohexyl, —CH2-cycloheptyl, or —CH2-cyclooctyl. The “cycloalkyl” can be optionally substituted. A “cycloheteroalkyl”, as used herein, unless otherwise indicated, can include any of the above with a carbon substituted with a heteroatom (e.g., O, S, N).

The term “heterocyclic” or “heteroaryl”, as used herein, unless otherwise indicated, can include an aromatic or non-aromatic cycloalkyl in which one to four of the ring carbon atoms are independently replaced with a heteroatom from the group consisting of O, S and N. Representative examples of a heterocycle include, but are not limited to, benzofuranyl, benzothiophene, indolyl, benzopyrazolyl, coumarinyl, isoquinolinyl, pyrrolyl, pyrrolidinyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl, (1,4)-dioxane, (1,3)-dioxolane, 4,5-dihydro-1H-imidazolyl, or tetrazolyl. Heterocycles can be substituted or unsubstituted. Heterocycles can also be bonded at any ring atom (i.e., at any carbon atom or heteroatom of the heterocyclic ring). A heterocyclic can be saturated, partially saturated, or unsaturated. The “hetreocyclic” can be optionally substituted.

The term “indole”, as used herein, is an aromatic heterocyclic organic compound with the formula C₈H₇N. It has a bicyclic structure, consisting of a six-membered benzene ring fused to a five-membered nitrogen-containing pyrrole ring. The “indole” can be optionally substituted.

The term “cyano”, as used herein, unless otherwise indicated, can include a —CN group. The “cyano” can be optionally substituted.

The term “alcohol”, as used herein, unless otherwise indicated, can include a compound in which the hydroxyl functional group (—OH) is bound to a carbon atom. In particular, this carbon center should be saturated, having single bonds to three other atoms. The “alcohol” can be optionally substituted.

The term “solvate” is intended to mean a solvate form of a specified compound that retains the effectiveness of such a compound. Examples of solvates include compounds of the invention in combination with, for example: water, isopropanol, ethanol, methanol, dimethylsulfoxide (DMSO), ethyl acetate, acetic acid, or ethanolamine.

The term “mmol”, as used herein, is intended to mean millimole. The term “equiv”, as used herein, is intended to mean equivalent. The term “mL”, as used herein, is intended to mean milliliter. The term “g”, as used herein, is intended to mean gram. The term “kg”, as used herein, is intended to mean kilogram. The term “μg”, as used herein, is intended to mean micrograms. The term “h”, as used herein, is intended to mean hour. The term “min”, as used herein, is intended to mean minute. The term “M”, as used herein, is intended to mean molar. The term “μL”, as used herein, is intended to mean microliter. The term “μM”, as used herein, is intended to mean micromolar. The term “nM”, as used herein, is intended to mean nanomolar. The term “N”, as used herein, is intended to mean normal. The term “amu”, as used herein, is intended to mean atomic mass unit. The term “° C.”, as used herein, is intended to mean degree Celsius. The term “wt/wt”, as used herein, is intended to mean weight/weight. The term “v/v”, as used herein, is intended to mean volume/volume. The term “MS”, as used herein, is intended to mean mass spectroscopy. The term “HPLC”, as used herein, is intended to mean high-performance liquid chromatography. The term “RT”, as used herein, is intended to mean room temperature. The term “e.g.”, as used herein, is intended to mean example. The term “N/A”, as used herein, is intended to mean not tested.

As used herein, the expression “pharmaceutically acceptable salt” refers to pharmaceutically acceptable organic or inorganic salts of a compound of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, or pamoate (i.e., 1,1′-methylene-bis-(2-hydroxy-3-naphthoate)) salts. A pharmaceutically acceptable salt may involve the inclusion of another molecule such as an acetate ion, a succinate ion, or another counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counterions. Hence, a pharmaceutically acceptable salt can have one or more charged atoms and/or one or more counterion. As used herein, the expression “pharmaceutically acceptable solvate” refers to an association of one or more solvent molecules and a compound of the invention. Examples of solvents that form pharmaceutically acceptable solvates include but are not limited to, water, isopropanol, ethanol, methanol, DMSO, ethyl acetate, acetic acid, and ethanolamine. As used herein, the expression “pharmaceutically acceptable hydrate” refers to a compound of the invention, or a salt thereof, that further can include a stoichiometric or non-stoichiometric amount of water bound by non-covalent intermolecular forces.

### Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refers to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs the transcription of a nucleic acid. An inducible promoter is generally understood as a promoter that mediates the transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site, all other sequences of the gene and its controlling regions can be numbered. Downstream sequences (i.e., further protein-encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects the expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid that is replaced has a similar property as the original amino acid, for example the exchange of Glu by Asp, GIn by Asn, Val by lie, Leu by lie, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. The amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in vitro using the specific codon usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41 (fraction G/C content)−0.63(% formamide)−(600/1). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated into the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| | |
|---|---|
| Side Chain Characteristic | Amino Acid |

Non-polar (hydrophobic)

| | |
|---|---|
| A. Aliphatic: | A L I V P |
| B. Aromatic: | F W |
| C. Sulfur-containing: | M |
| D. Borderline: | G |

Uncharged-polar

| | |
|---|---|
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids which may be introduced to a host cell include, for example, DNA sequences or genes from another species, or even genes or sequences which originate with or are present in the same species but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

### Genome Editing

As described herein, lncRNA signals including but not limited to LINC01432 can be modulated (e.g., reduced, eliminated, or enhanced) using genome editing. Processes for genome editing are well known; see e.g. Aldi 2018 Nature Communications 9(1911). Except as otherwise noted herein, therefore, the process of the present disclosure can be carried out in accordance with such processes.

For example, genome editing can comprise CRISPR/Cas9, CRISPR-Cpf1, TALEN, or ZNFs. Adequate blockage of an lncRNA such as LINC01432 by genome editing can result in protection from autoimmune or inflammatory diseases.

As an example, clustered regularly interspaced short palindromic repeats (CRISPR)/CRISPR-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N)₂₀NGG target DNA sequence). This results in a double-strand break three nucleotides upstream of the NGG motif. The double strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome. Thus, genomic editing, for example, using CRISPR/Cas systems could be useful tools for therapeutic applications for multiple myeloma to target cells by the removal of LINC01432 signals.

For example, the methods as described herein can comprise a method for altering a target polynucleotide sequence in a cell comprising contacting the polynucleotide sequence with a clustered regularly interspaced short palindromic repeats-associated (Cas) protein.

### Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption-delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of the agent being metabolized or excreted from the body. The controlled release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for the treatment of the disease, disorder, or condition.

### Therapeutic Methods

Also provided is a process of treating, preventing, or reversing multiple myeloma in a subject in need of administration of a therapeutically effective amount of an lncRNA modulation agent, so as to inhibit LINC01432.

Methods described herein are generally performed on a subject in need thereof. A subject in need of the therapeutic methods described herein can be a subject having, diagnosed with, suspected of having, or at risk for developing multiple myeloma. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of an lncRNA modulating agent is, for example, an amount that would cause the desired therapeutic effect in a subject while minimizing undesired side effects. In various embodiments, an effective amount of an lncRNA modulating agent described herein can substantially inhibit multiple myeloma, slow the progress of multiple myeloma, or limit the development of multiple myeloma.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of an lncRNA modulating agent can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to inhibit LINC01432.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD50 (the dose lethal to 50% of the population) and the ED50, (the dose therapeutically effective in 50% of the population). The dose ratio between toxic and therapeutic effects is the therapeutic index that can be expressed as the ratio LD50/ED50, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single-dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of an lncRNA modulating agent can occur as a single event or over a time course of treatment. For example, an lncRNA modulating agent can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accordance with the methods described herein can be performed prior to, concurrent with, or after conventional treatment modalities for multiple myeloma.

An lncRNA modulating agent can be administered simultaneously or sequentially with another agent, such as an antibiotic, an anti-inflammatory, or another agent. For example, an lncRNA modulating agent can be administered simultaneously with another agent, such as an antibiotic or an anti-inflammatory. Simultaneous administration can occur through administration of separate compositions, each containing one or more of an lncRNA modulating agent, an antibiotic, an anti-inflammatory, or another agent. Simultaneous administration can occur through administration of one composition containing two or more of an lncRNA modulating agent, an antibiotic, an anti-inflammatory, or another agent. An lncRNA modulating agent can be administered sequentially with an antibiotic, an anti-inflammatory, or another agent. For example, an lncRNA modulating agent can be administered before or after administration of an antibiotic, an anti-inflammatory, or another agent.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well-known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

### Screening

Also provided are methods for screening.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example, ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character xlogP of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of compounds during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

### Kits

Also provided are kits. Such kits can include an agent or composition described herein and, in certain embodiments, instructions for administration. Such kits can facilitate the performance of the methods described herein. When supplied as a kit, the different components of the composition can be packaged in separate containers and admixed immediately before use. Components include, but are not limited to an lncRNA modulating agent, solubilizers, and clinical multiple myeloma drugs such as melphalan. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing the activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet website specified by the manufacturer or distributor of the kit.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

The methods and algorithms of the invention may be enclosed in a controller or processor. Furthermore, methods and algorithms of the present invention can be embodied as a computer-implemented method or methods for performing such computer-implemented method or methods, and can also be embodied in the form of a tangible or non-transitory computer-readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer programs include, for example, floppy disks and diskettes, compact disk (CD)-ROMs (whether or not writeable), DVD digital disks, RAM and ROM memories, computer hard drives and back-up drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—LINC01432 Binds to CELF2 in Newly Diagnosed Multiple Myeloma Promoting Poor Response to Standard Therapy

Multiple Myeloma (MM) is a highly prevalent and incurable form of cancer that arises from malignant plasma cells, with over 35,000 new cases diagnosed annually in the United States. While there are a growing number of approved therapies, MM remains incurable, and nearly all patients will relapse and exhaust all available treatment options. Mechanisms for disease progression are unclear and in particular, little is known regarding the role of long non-coding RNAs (lncRNA) in mediating disease progression and response to treatment. In this study, we used transcriptome sequencing to compare newly diagnosed MM patients who were poor responders to standard first-line treatment (progression free survival <24 months) to patients who were standard responders (progression free survival >24 months). We identified 86 differentially upregulated lncRNAs in poor responders and focused our efforts on characterizing the most upregulated lncRNA, LINC01432.

### Introduction

Multiple myeloma (MM) is a prevalent disease; and it is the fifteenth leading cause of cancer-related death in the United States. Despite the increasing availability of treatment regimens, nearly all patients with MM become refractory and die from the disease or its sequela. In addition, while some improvements in patient outcomes have been achieved using novel immunomodulatory agents, new approaches are still needed due to high toxicity and the development of drug resistance. This underscores the need for new therapeutic approaches. Thus, knowledge surrounding the mechanisms and biomarkers of treatment resistance in MM patients are critically needed to support the development of novel MM therapies.

Long non-coding RNA (lncRNA) is defined as RNA greater than 200 nucleotides in length that is not translated into functional proteins. Prior studies have reported that lncRNAs can promote the pathogenesis of all cancer types, including MM. Many lncRNAs have also been shown to promote MM drug resistance, including NEAT1, ANRIL, MEG3, LINC00461, H19, and PCAT1. lncRNAs are expressed in the cytoplasm, the nucleus, and in other organelles, such as exosomes, and may be expressed in more than one subcellular location. The subcellular localization of a lncRNA is highly important and specific to its biological functions in the cell, which may include transcriptional regulation, translational regulation, and interaction with RNA binding proteins. As lncRNA expression is highly tissue specific, they hold promise as novel therapeutic targets that can be used as prognostic and diagnostic biomarkers. Further, recent advances in understanding the functions and crucial roles lncRNAs play in promoting cancer, including MM, increases their potential as targets for RNA-based therapeutics.

Limited availability of RNA sequencing or single cell sequencing data from newly diagnosed multiple myeloma (NDMM) patients, along with the heterogeneity of treatment approaches used with MM patients, have hindered research on the global expression of lncRNAs in MM and characterization of their biological functions in response to current standard MM therapies. In this study, we used RNA sequencing data from a cohort of NDMM patients to identify lncRNAs that were associated with a poor response. We identified several lncRNAs that were highly upregulated in Poor Responders, as compared to Standard Responders, and determined that LINC01432 bound to the RNA-binding protein, CELF2, to inhibit apoptosis and increase viability.

### Results

### Identification of Dysregulated LncRNAs in Poor Responders to Standard Mm Therapy.

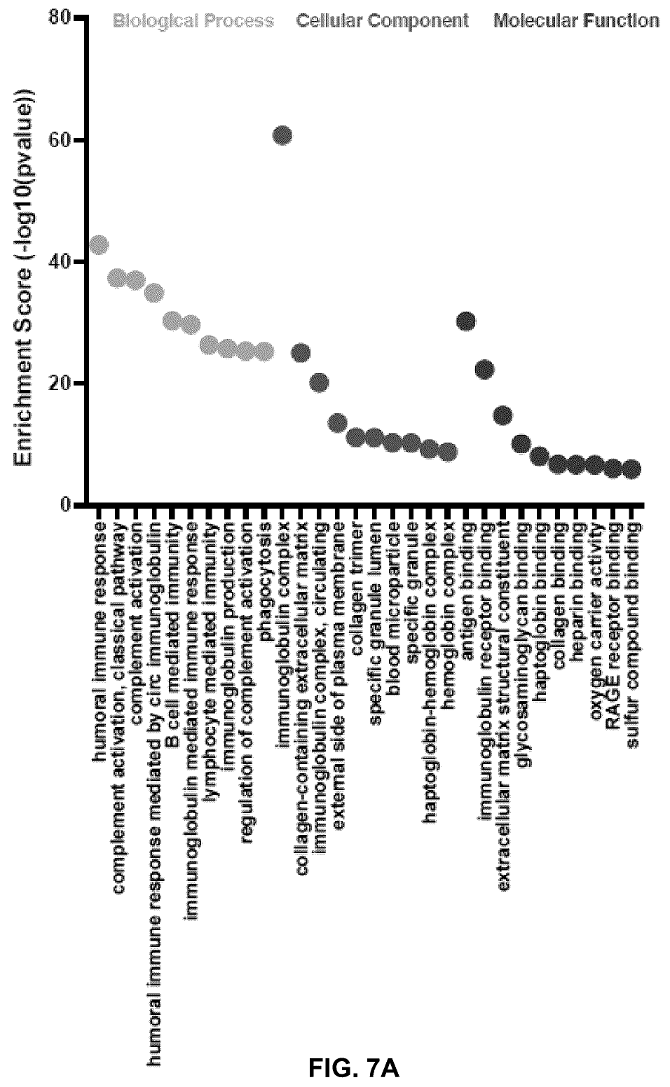

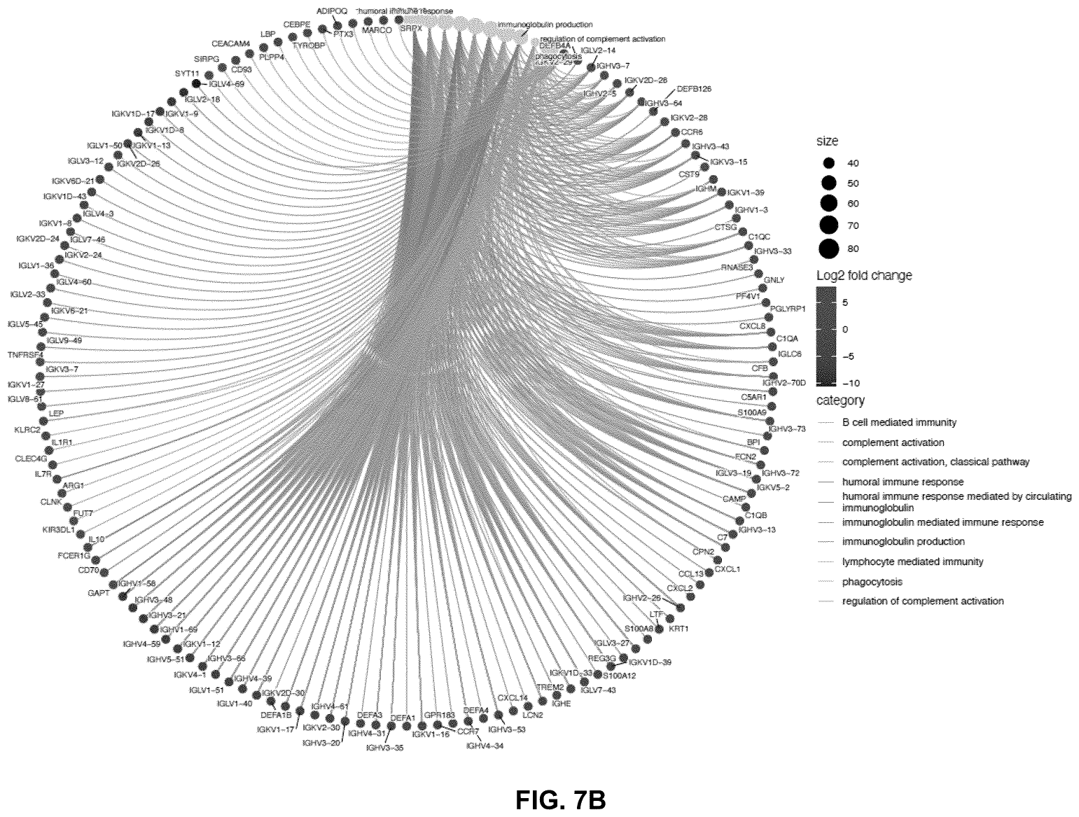

In order to identify lncRNAs that are differentially expressed in patients that exhibit a poor response to the standard MM treatment approach, we analyzed transcriptome sequencing data from CD138+ bone marrow samples obtained from 116 NDMM patients in the MMRF CoMMpass study. We assigned samples to one of two groups based on each patient's response to standard MM therapy, Standard Responders, those who had progression free survival >24 months from first dose of MM treatment (N=78), and Poor Responders, those with <24 months progression free survival (N=38) (Table 1). We identified 86 upregulated and 158 downregulated lncRNAs in Poor Responders, as compared to Standard Responders (log 2Fold Change >+/−2, p value <0.05), (FIG. 1A). lncRNAs identified as being most differentially expressed in Poor Responders included LINC01432, Inc-LGALS9B-7, LINC01916, Lnc-SPIDR-1, and MAGEA4-AS1, (FIG. 1B). We also identified two lncRNAs previously reported to be associated with MM, MEG324,50,51 and H19,52-55. Next, we performed pathway analysis on all differentially expressed RNAs in Poor Responders to identify highly enriched gene sets associated with, but not limited to, Staphylococcus aureus infection (p=6.75e-10), transcriptional dysregulation in cancer (p=1.05e-06), cytokine-cytokine receptor interactions (p=1.42e-05), IL-17 signaling pathway (p=2.31e-05), and ECM-receptor interactions (p=5.5e-05), (FIG. 1C). Gene ontology analysis further showed high enrichment of more than one pathway associated with immune response, B cell mediated immunity, and multiple hemoglobin complexes, (FIG. 7A, FIG. 7B). This analysis of sequencing data from NDMM patients in the MMRF CoMMPass study allowed us to identify lncRNAs that are differentially expressed in patients that exhibit a poor response to standard MM treatment.

TABLE 1
Demographic details of NDMM patients separated
into Poor Responders and Standard Responders
to standard multiple myeloma therapy

| | No. of Patients (%) | | |
|---|---|---|---|
| | Poor Responders | Standard Responders | Total |
| Characteristics | (n = 38) | (n = 77) | 115 |

Age

| | | | | | |
|---|---|---|---|---|---|
| ≤60 | 23 | (33.3) | 46 | (66.7) | 69 |
| ≥60 | 15 | (32.6) | 31 | (67.4) | 46 |

Gender

| | | | | | |
|---|---|---|---|---|---|
| Male | 22 | (37.9) | 36 | (26.1) | 58 |
| Female | 16 | (28.1) | 41 | (71.9) | 57 |

Race

| | | | | | |
|---|---|---|---|---|---|
| White | 31 | (34.8) | 58 | (65.2) | 89 |
| AA black | 4 | (22.2) | 14 | (77.8) | 18 |
| Other | 3 | (37.5) | 5 | (62.5) | 8 |

Feature

| | | | | | |
|---|---|---|---|---|---|
| Del17p | 1 | (100) | 0 | (0) | 1 |
| Amp1q | 17 | (47.2) | 19 | (52.8) | 36 |
| t4:14 | 5 | (45.5) | 6 | (54.5) | 11 |
| t8:14 | 10 | (50.0) | 10 | (50.0) | 20 |
| t11:14 | 4 | (23.5) | 13 | (76.5) | 17 |
| t14:16 | 1 | (33.3) | 2 | (66.7) | 3 |
| t14:20 | 0 | (0) | 0 | (0) | 0 |

R-ISS

| | | | | | |
|---|---|---|---|---|---|
| Stage I | 10 | (24.3) | 31 | (75.6) | 41 |
| Stage II | 23 | (35.3) | 42 | (64.6) | 65 |
| Stage III | 5 | (55.5) | 4 | (44.4) | 9 |

### LINC01432 is the Topmost Significant Upregulated LncRNA in Poor Responders.

We focused our subsequent experimental analyses on characterizing the most significantly upregulated lncRNA in Poor Responders, as compared to Standard Responders, termed LINC01432 (Fold change=6.37, p=1.23e⁻⁴¹) (FIG. 1B, FIG. 2A). LINC01432 is a long intergenic non-protein coding RNA located on chromosome 20, has four exons, and is 693 nucleotides long. There is little-to-no current knowledge about LINC01432; it has only been reported to contain a SNP associated with male baldness in a single-trait genome-wide association study. Due to the heterogeneity and hyperploidy in several chromosomes observed in MM patients, we began by characterizing LINC01432 by assessing different genetic subtypes of MM. We found that high expression of LINC01432 was correlated with t(14; 16) and Amp (1q) translocations (t[14; 16] positive correlation=0.57; Amp [1q] positive correlation=0.14) (FIG. 2B).

To further characterize LINC01432, we analyzed its expression in a panel of MM cell lines and found that LINC01432 is highly expressed in RPMI 8226 and OPM-2 cells, with low level expression detected in MM.1S, MM.1R, and U266B1 cells (FIG. 8A). Next, we confirmed expression of LINC01432 in NDMM bone marrow aspirates using mFISH (FIG. 2C). To assess the clinical significance of LINC01432 in the context of MM, we subcutaneously injected mice with the MM cell lines RPMI 8226 and U266B1 to assess in vivo tumor growth and LINC01432 expression. We found high expression of LINC01432 in RPMI 8226 tumors and low expression in U266B1 tumors using mFISH (FIG. 2D). We determined that LINC01432 is localized in both the cytoplasm and the nuclear compartments of RPMI 8226 cell line tumors, with 9.50% of cells exhibiting expression in nucleus, 0.59% exhibiting expression in the cytoplasm, 86.91% exhibiting expression in both compartments, and 2.99% with no apparent LINC01432 expression (FIG. 2E). In the U266B1 cell line, which has low endogenous LINC01432 expression levels, LINC01432 expression was located in the nucleus in 32.79% of cells, in the cytoplasm of 0.44% of cells, in both compartments of 15.83% of cells, and expression was not detected in 50.94% of cells (FIG. 2F). These data indicate that LINC01432 is highly expressed in NDMM patient samples and in MM cell lines and is a novel lncRNA expressed in patients with poor response to standard treatment.

### LINC01432 Inhibits Apoptosis and Increases Tumor Growth.

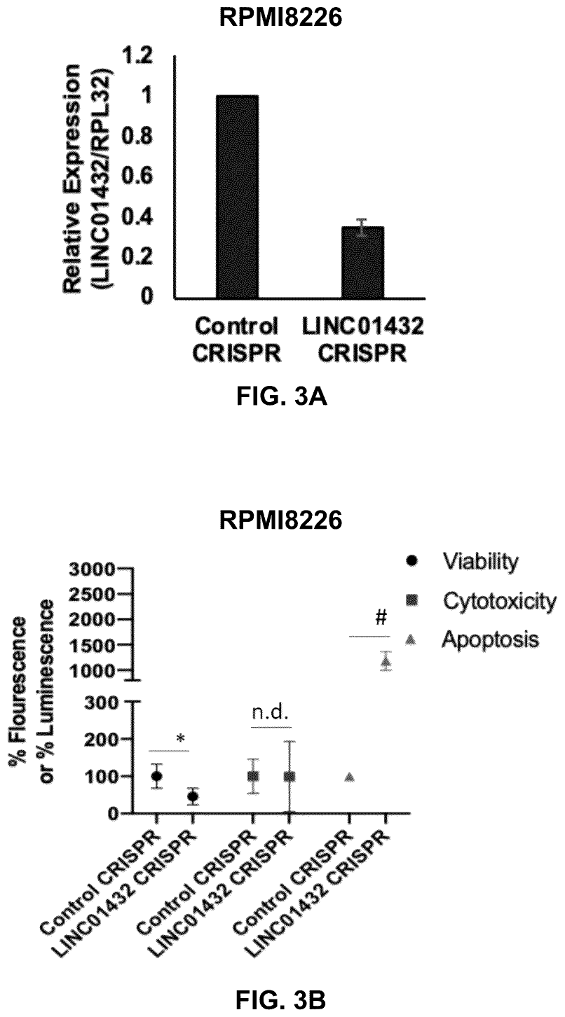

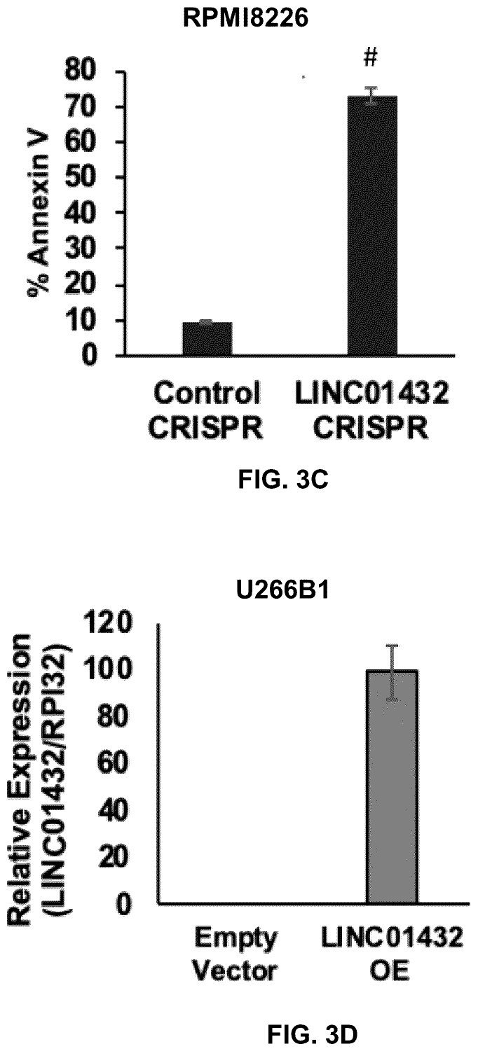

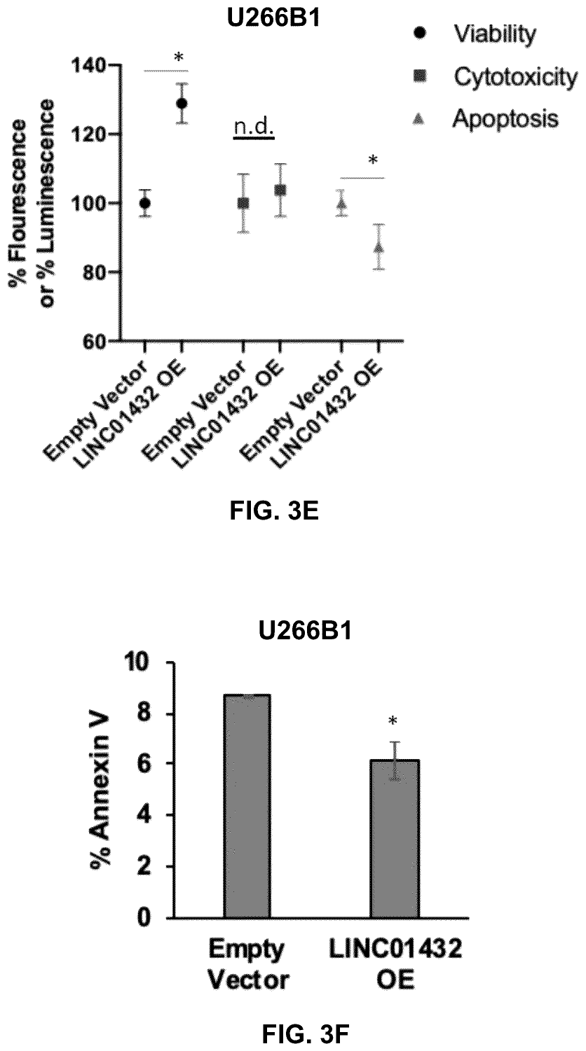

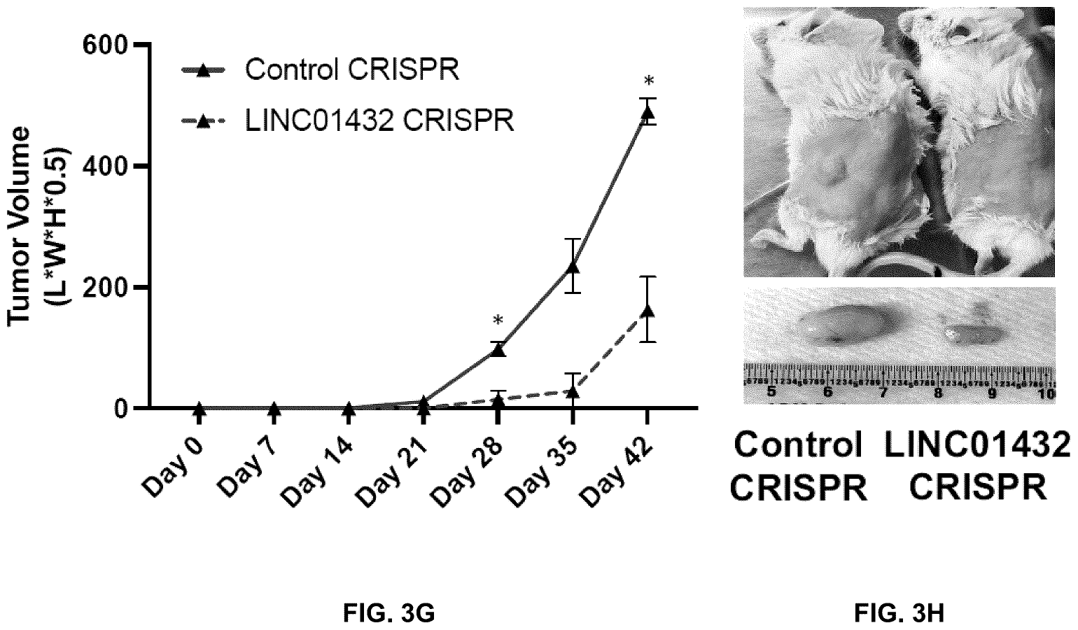

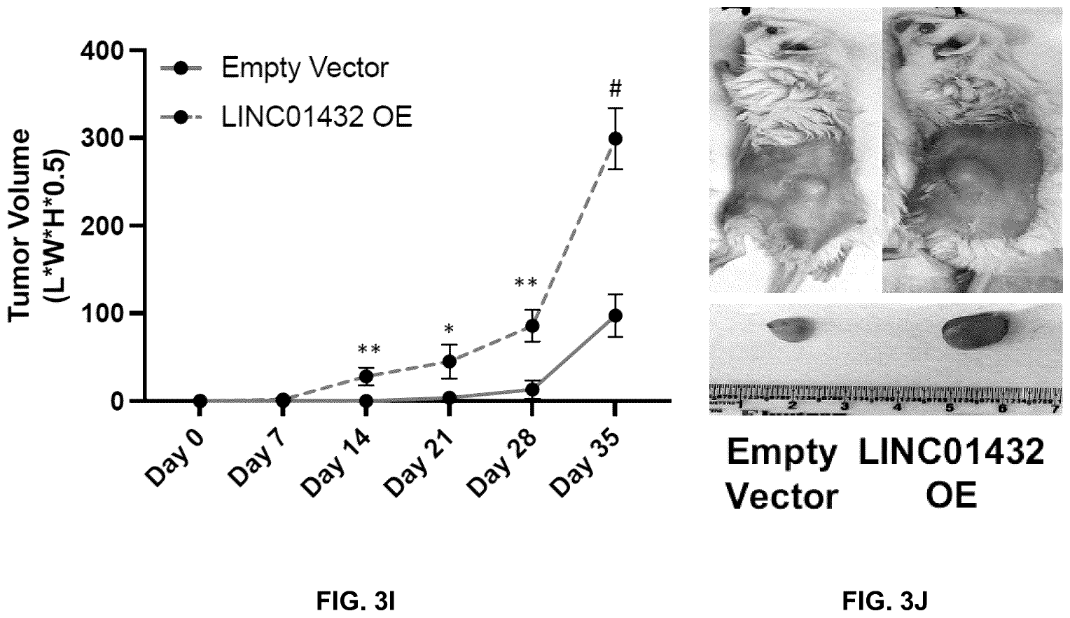

To investigate the molecular mechanisms through which LINC01432 may induce a poor response to standard MM therapy and to test its potential as a therapeutic target, we used CRISPR/Cas9 (CRISPR) to knockdown LINC01432 expression in RPMI 8226 cells that have high endogenous expression levels (FIG. 3A, FIG. 8A). Using the ApoTox-Glo Triplex Assay, we found that LINC01432 knockdown significantly decreased viability (p=0.03) and significantly increased apoptosis (p=2.69e⁻⁰⁵) in these cells, as compared to control CRISPR cells (FIG. 3B). Increased apoptosis in LINC01432 knockdown cells was further validated via Annexin V flow cytometry (p=1.25e⁻⁰⁶) (FIG. 3C). We then generated LINC01432 overexpression cells from U266B1 cells, which have low endogenous LINC01432 expression levels (FIG. 3D, FIG. 8A) and found that these cells have significantly increased viability (p=0.001) and significantly decreased apoptosis (ApoTox-Glo p=0.04, AnnexinV p=0.04), as compared to empty vector control cells (FIG. 3E, FIG. 3F). Next, we subcutaneously injected mice with LINC01432 knockdown and control CRISPR cell lines and compared in vivo tumor growth. This revealed significantly lower tumor volume in mice injected with LINC01432 knockdown cells, as compared to control cells (Day 28 p=0.04, Day 42 p=0.02) (FIG. 3G, FIG. 3H). We similarly injected mice with LINC01432 overexpression cells and empty vector controls and found that overexpression resulted in significantly higher tumor volume, as compared to controls (Day 14 p=0.009, Day 21 p=0.04, Day 28 p=0.003, Day 35 p=0.000) (FIG. 3I, FIG. 3J).

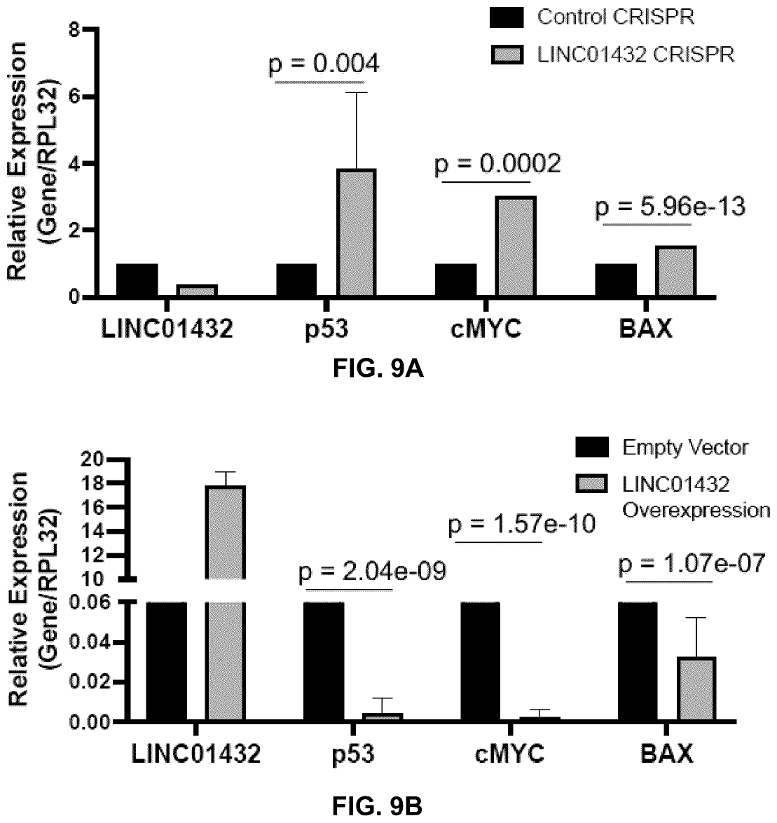

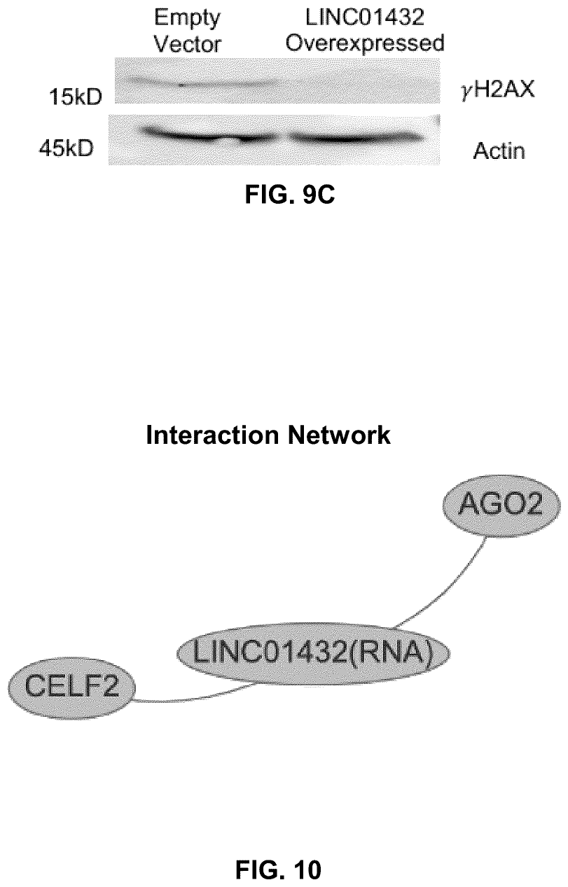

Next, we assessed the effects of LINC01432 knockdown and overexpression on apoptotic markers, including TP53 pathway genes, via RT-qPCR. We found that expression of apoptotic markers was significantly higher in RPMI 8226 LINC01432 knockdown cells, as compared to controls (TP53p=0.004, cMYC p=0.0002, BAX p=5.96e⁻¹³). Similarly, we found that expression of apoptotic markers was significantly lower in tumors arising from LINC01432 overexpression cells, as compared to controls (TP53p=2.04e⁻⁰⁹, cMYC p=1.57e⁻¹⁰, BAX p=1.07e⁻⁰⁷) (FIG. 9A, FIG. 9B). In addition, we detected a decrease in γH2AX, a marker of DNA double-stranded breaks, in tumors arising from LINC01432 overexpression cells (FIG. 9C). These data provide evidence that LINC01432 is highly expressed in MM cell lines and its expression is associated with increased viability and decreased apoptosis.

### LINC01432 Binds to CELF2 Protein.

Many functional studies of lncRNAs, including our group's research, have found that the ability of lncRNAs to bind with proteins and regulate downstream genes is integral to their roles in cancer and therapeutic resistance. As limited functional data on LINC01432 is available, we utilized POSTAR3 as a first step to identifying proteins which potentially bind to LINC01432. POSTAR3 is a unique, comprehensive database of post-transcriptional regulation and RNA-binding proteins that incorporates publicly available large-scale datasets on CLIP-sequencing, Ribo-sequencing, RNA secondary structure, and miRNA-mediated degradation events. This analysis identified just two RNA-binding proteins in publicly available CLIP-sequencing datasets that are known to bind to LINC01432, CELF2 (identified in T-cells) and AGO2 (identified in cardiac tissue) (FIG. 10). Although the determined binding score was low (0.019) for both proteins, CELF2 (CUGBP Elav-like family) proteins are RNA-binding proteins with pleiotropic capabilities in RNA processing that have been found to compete with non-coding RNAs, including lncRNAs. CELF2 has been shown to bind lncRNAs to regulate downstream mRNAs, thereby promoting proliferation, migration, and tumor growth of multiple cancers, however, this has not yet been studied in MM. Thus, we investigated whether LINC01432 binds to CELF2. Future studies will determine the importance of LINC01432 binding to AGO2 protein.

Analysis of our NDMM patient RNA sequencing dataset revealed high level expression of CELF2 (log CPM >50), but no significant differences in expression levels were identified between Poor Responders and Standard Responders (FIG. 4C). The Human Protein Atlas (proteinatlas.org) indicates that CELF2 expression is enriched in bone marrow (Tau score=0.40) and localized to the nucleoplasm, vesicle, and midbody ring. Assessment of CELF2 expression in multiple blood cancer types indicated that CELF2 is highly expressed in leukemia, lymphoma, and MM (log CPM >10) (FIG. 11A). Western blot analysis of CELF2 protein expression in whole cell lysates showed slight increased expression of CELF2 in both LINC01432 RPMI 8226 knockdown (Fold Change=1.41) and U266B1 overexpression cells (Fold Change=1.58), as compared to controls (FIG. 11B, FIG. 11C). mFISH analysis using LINC01432 probes in combination with CELF2 protein immunohistochemistry in MM cells indicated that CELF2 is expressed in both the nucleus and cytoplasm (FIG. 4D, FIG. 4E). Further, we found evidence of CELF2 and LINC01432 co-localization in both cellular compartments in RPMI 8226 wild-type cells and U266B1 LINC01432 overexpression cells (FIG. 4F, FIG. 4G).

Next, we conducted iCLIP analysis to identify the regions of LINC01432 that may be directly bound by CELF2, which combines UV cross-linking with immunoprecipitation and RT-qPCR to precisely map the binding sites of RNA-binding proteins (FIG. 4H). RT-qPCR tiling primers spanning LINC01432 showed direct binding of CELF2 to Tiling Primer 1 (Fold Change >2), Tiling Primer 4 (Fold Change >8), and Tiling Primer 5 (Fold Change >8), as compared to IgG negative control, in RPMI 8226 cells with high level endogenous expression of LINC01432 (FIG. 4I, FIG. 4J). These data indicate that LINC01432 is bound by CELF2 protein in MM cell lines.

Treating Cells with LINC01432 Locked Nucleic Acid Antisense Oligo (LNA ASOs) Increased Apoptosis.

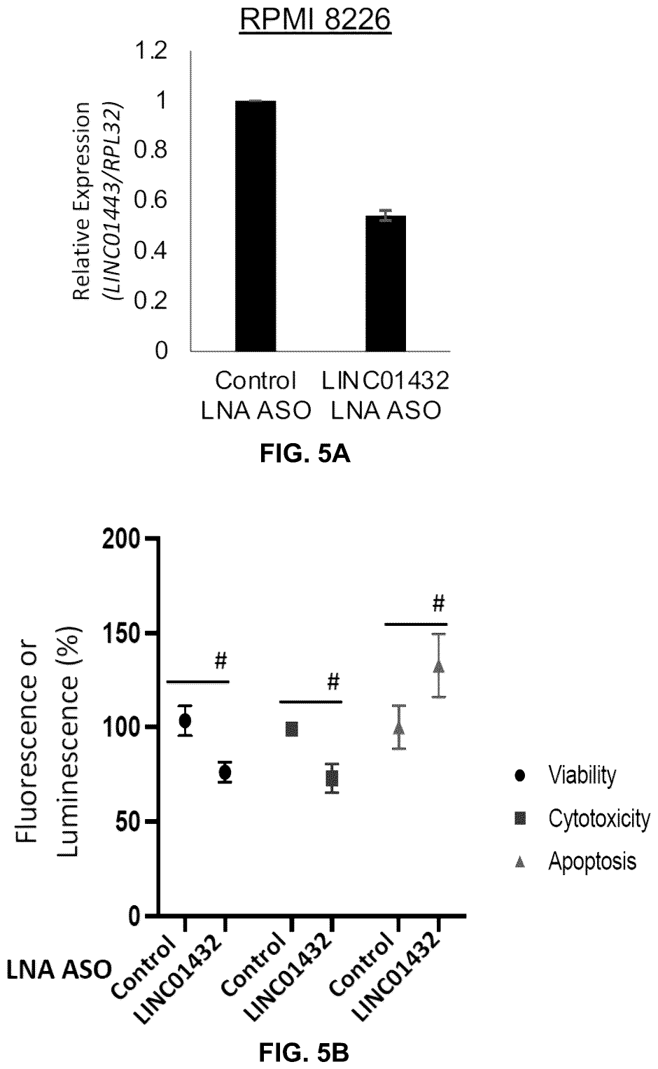

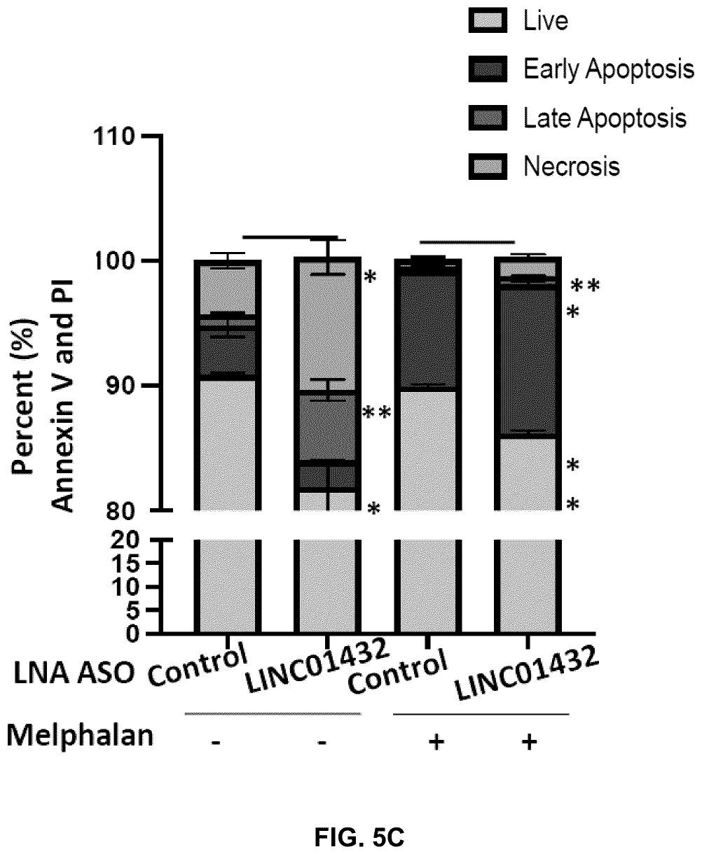

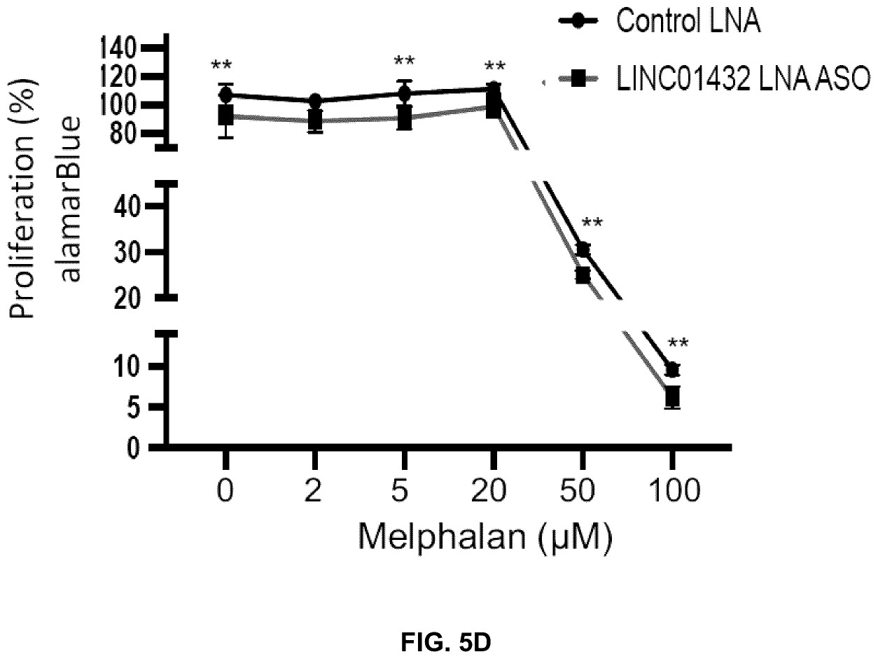

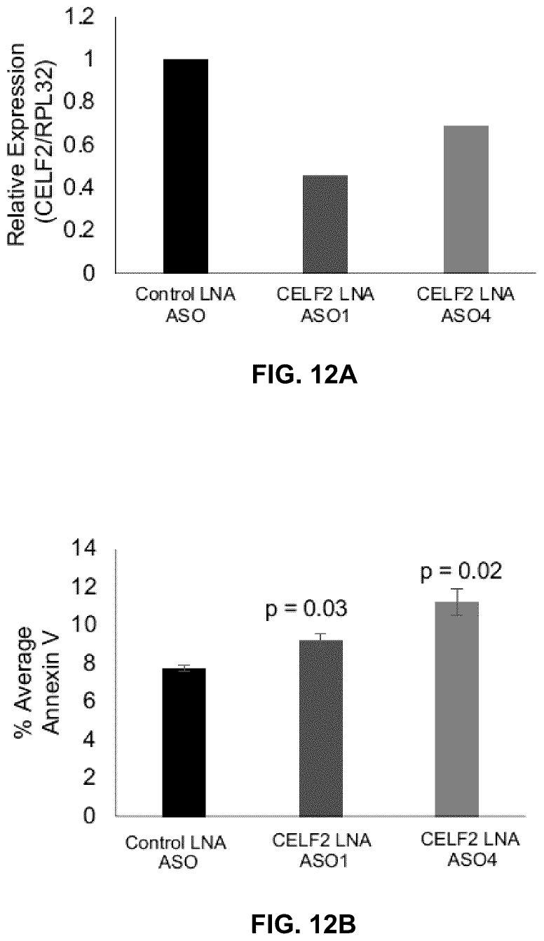

To assess the potential of LINC01432 as a therapeutic target, we developed LINC01432-targeted LNA ASOs, which are increasingly being evaluated in clinical trials, along with control LNA ASOs. We treated RPMI 8226 cells with these LNA ASOs and confirmed knockdown of LINC01432 expression (FIG. 5A). We also showed that LNA ASO-mediated LINC01432 knockdown induced significant decreases in viability (p=0.001) and cytotoxicity (p=0.0005), and a significant increase in apoptosis (p=0.017) (FIG. 5B). We then treated RPMI 8226 cells with LINC01432 or control LNA ASOs followed by 24 hours of 30 μM Melphalan treatment and found that LNA ASO-mediated LINC01432 knockdown increased the proportion of cells in early apoptosis and necrosis, as measured by flow cytometry (FIG. 5C), and decreased proliferation, as compared to control LNA ASO treatment (FIG. 5D). Further, we showed that LNA ASO-mediated CELF2 knockdown increased apoptosis in MM cell lines (FIG. 12A, FIG. 12B). These data provide evidence that LINC01432 inhibits apoptosis and LNA ASO-mediated knockdown of LINC01432 can induce sensitivity to melphalan.

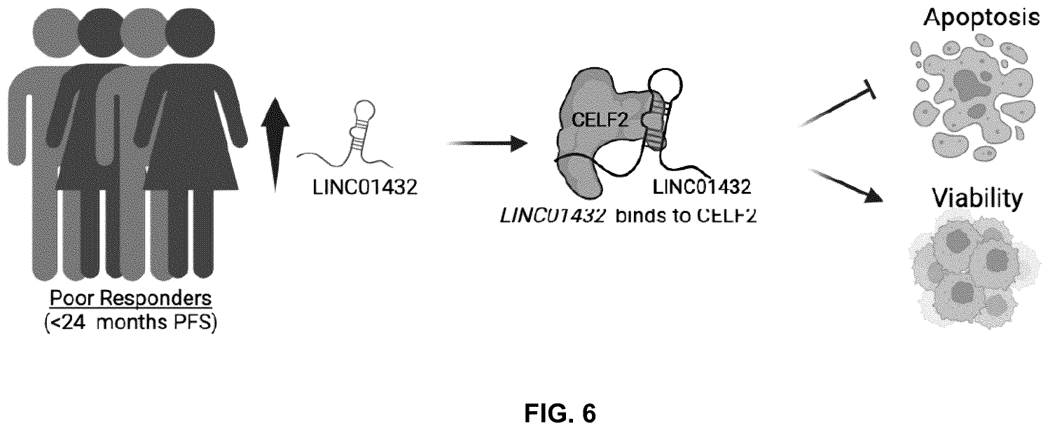

In summary, our study identified differentially expressed lncRNAs associated with patients who were Poor Responders to standard MM therapy and determined that LINC01432 bound to CELF2 and that ASO mediated knockdown of either in RPMI 8226 cells promotes apoptosis and decreases viability suggesting the role of LINC01432 and the LINC01432-CELF2 complex in promoting resistance to chemotherapy (FIG. 6).

### Discussion

The development of drug resistance, results in most patients exhausting all available treatment options and relapsing. While some advances in MM treatment are emerging through clinical trials of novel cellular immunotherapies targeting immune cells, including chimeric antigen receptor T cell therapies, unfortunately, interpatient heterogeneity has hindered the elucidation of the molecular mechanisms that control progression of plasma cells in patients with MM. Thus, knowledge surrounding mechanisms and biomarkers related to treatment resistance in MM patients are critical for novel therapy development.

In this study, we identified differentially expressed lncRNAs in NDMM patients who exhibited a poor response to the standard MM therapy. The most upregulated annotated lncRNA, LINC01432, was found to bind to the CELF2 protein, leading to inhibition of apoptosis and promotion of cell viability. CELF2 has been previously reported to bind to lncRNAs and regulate downstream mRNAs, thereby promoting proliferation, migration, and tumor growth in multiple forms of cancer, however, this has not yet been studied in the context of NDMM. Interestingly, CELF2 expression patterns vary in different developmental and differentiation stages. In cancer, CELF2 has been found to be localized to the nucleus, where it is associated with alternative splicing and transcript editing, in RNA granules, where it regulates mRNA stability, and in the cytoplasm, where it regulates pre-miRNA maturation, translation, and alternative polyadenylation. Here, we determined that CELF2 shows different patterns of expression in MM cell lines with differential levels of endogenous LINC01432 expression. In the presence of LINC01432, CELF2 was localized to the cytoplasm. This co-localization allowed binding of LINC01432 to CELF2 to inhibit apoptosis. As we are in the earliest stages of understanding LINC01432 tumor biology, this study allows us to predict that LINC01432-CELF2 interaction may play a larger role in the pathogenesis of MM. Future studies are needed to better understand this interaction in MM and to fully characterize its role in the development of resistance to chemotherapy.

One promising aspect of lncRNAs that makes them ideal novel targets for the development of RNA therapeutics is their tissue and cell specific expression patterns. ASOs are an emerging class of RNA-based therapeutic drugs that can be easily modified and optimized for clinical development. To date, there are 128 registered clinical trials of ASOs for the treatment of several diseases, including cancer. ASOs have been shown to be a powerful tool for therapeutically targeting lncRNAs. We developed a LINC01432-targeted LNA ASO and demonstrated its potential use to treat LINC01432-mediated decreased apoptosis and increased viability in in vitro MM cell lines. Further, co-treating MM cell lines with LINC01432-targeted LNA ASO and high-dose melphalan enhanced sensitivity to melphalan and increased apoptosis in vitro. This study represents a preliminary investigation into the use of LNA ASO to downregulate LINC01432 lncRNA; future studies are being conducted to provide evidence of its clinical significance.

Many unanswered questions remain regarding the exact mechanism through which LINC01432 regulates downstream pathways and mediates epigenetic regulation while bound to CELF2. In conclusion, our study provides preliminary insights into the role of lncRNA expression in NDMM patients who exhibit a poor response to standard MM therapy and identifies a novel potential target for the development of future MM therapies.

### Methods

### RNA Sequencing Data, Patient Samples, and Cell Lines.

RNA sequencing data from NDMM patients were obtained from the Multiple Myeloma Research Foundation (MMRF) Clinical Outcomes in Multiple Myeloma to Personal Assessment of Genetic Profiles (CoMMpass) study (https://registry.opendata.aws/mmrf-commpass). MM cell lines were generously provided by Dr. John DiPersio at Washington University in St. Louis (RPMI 8226, U266B1, MM1.S, and OPM2) and were all cultured in RPMI 1640 media (Invitrogen, Carlsbad, CA) supplemented with 15% fetal bovine serum (Invitrogen) and 1% penicillin/streptomycin (Invitrogen). MM1.R cell lines were purchased from ATCC (catalog number CRL-2975) and cultured in the same manner as the other cell lines. NDMM patient bone marrow aspirates were obtained from the Multiple Myeloma Tissue Banking Protocol (IRB 201102270) processed by the Siteman Cancer Center Tissue Procurement Core.

Full length LINC01432 transcript was amplified via PCR and cloned into the pCFG5-IEGZ-GFP vector to create the pCFG5-IEGZ-GFP-Luc-LINC01432 vector (pCFG5-LINC01432). Full vector length was confirmed by GeneScript. Retroviral infection of HEK 293T cells was performed by transfecting cells with 2 μg of empty vector control or pCFG5-LINC01432. Transduction was conducted by harvesting viral supernatants and adding to U266B1 cells in the presence of 8 μg/ml polybrene (Sigma), then centrifuged at 500 g for three hours. Fresh media was then added, and cells were sorted for positive GFP expression via flow cytometry. Cells containing virus expressing LINC01432 or empty vector were selected for using 100 μg/ml Zeocin. Validated cell lines showing high levels of LINC01432 expression by RT-qPCR, as compared to empty vector, were used for subsequent assays.

LINC01432 knockdown CRISPR/Cas9 cells were generated using the RPMI 8226 cell line. The sgRNAs were generated by the Genome Engineering and Stem Cell Center, Washington University in St. Louis. sgRNAs were cloned into the pLV hUbC-dCas9 KRAB-T2A-GFP plasmid (Addgene #672620). HEK 293T cells were infected with this lentivirus to induce expression of dCas9-KRAB, followed by transduction, similar as above into RPMI 8226 cells and validated knockdown of LINC01432 expression via RT-qPCR.

### Transfection of Locked Nucleic Acid Antisense Oligonucleotides.

Locked nucleic acid GapmeR antisense oligonucleotides (LNA ASOs) targeting LINC01432 (Qiagen, cat #3653410) and CELF2 (Qiagen, cat #339511), and negative control LNA ASOs (Qiagen, cat #148759394), were designed using the Qiagen Antisense LNA GapmeR Custom Builder (https://www.qiagen.com), sequences are listed in Table 2. RPMI 8226 cells were seeded at a density of 500,000 cells/well in 6-well plates, transfected with respective ASOs at 100 nM concentration using Lipofectamine 2000, and incubated for 48-72 hours. Cells were harvested and target knockdown was validated via RT-qPCR.

TABLE 2
Primer sequences

| Primer | | | |
|---|---|---|---|
| name | Forward | Reverse | Application |
| LINC01432 | TGATCACTGGCACCATCACT | TAAAGCCATTCGCCTTCC | RT-qPCR |
| | (SEQ_ID_NO: 1) | TA (SEQ_ID_NO: 2) | |
| CELF2 | AATGCTCTCAGGTATGGCGG | ATTCCTGAGTAGGCCTGG | RT-qPCR |
| | (SEQ_ID_NO: 3) | GT (SEQ_ID_NO: 4) | |
| GAPDH | ACTTTGTCAAGCTCATTTCC | CACAGGGTACTTTATTGA | RT-qPCR |
| | (SEQ_ID_NO: 5) | TG (SEQ_ID_NO: 6) | |
| Actin | CTCGACACCAGGGCGTTATG | CCACTCCATGCTCGATAG | RT-qPCR |
| | (SEQ_ID_NO: 7) | GAT (SEQ_ID_NO: 8) | |
| BAX | TGATGGACGGGTCCGGG | CAAAAGGGCCCCTGTCT | RT-qPCR |
| | (SEQ_ID_NO: 9) | TCA (SEQ_ID_NO: 10) | |
| TP53 | TGACACGCTTCCCTGGATTG | GCTCGACGCTAGGATCT | RT-qPCR |
| | (SEQ_ID_NO: 11) | GAC (SEQ_ID_NO: 12) | |
| PUMA | AGGATGAAATTTGGCATGGGG | TCCCTGGGGCCACAAAT | RT-qPCR |
| | (SEQ_ID_NO: 13) | CT (SEQ_ID_NO: 14) | |
| MYC | CCCTCCACTCGGAAGGACTA | GCTGGTGCATTTTCGGTT | RT-qPCR |
| | (SEQ_ID_NO: 15) | GT (SEQ_ID_NO: 16) | |
| RPL32 | AGGCATTGACAACAGGGTTC | GTTGCACATCAGCAGCA | RT-qPCR |
| | (SEQ_ID_NO: 17) | CTT (SEQ_ID_NO: 18) | |
| LINC01432- | TGGAGAGAGTAAGGCGCATC | CCTGGGAGTGCAGGGTT | RT-qPCR |
| tile1 | (SEQ_ID_NO: 19) | TAT (SEQ_ID_NO: 20) | |
| LINC01432- | ATGACAGCTTGGAGCATCTG | TGGAGATTTTGCCATTTT | RT-qPCR |
| tile2 | (SEQ_ID_NO: 21) | CA (SEQ_ID_NO: 22) | |
| LINC01432- | TAGGAAGGCGAATGGCTTTA | CCATGGCCACAAGAGTT | RT-qPCR |
| tile3 | (SEQ_ID_NO: 23) | ACC (SEQ_ID_NO: 24) | |
| LINC01432- | CTGCCATGCAGAGGAGAAG | TGGCCTTTCCTTTATGCA | RT-qPCR |
| tile4 | (SEQ_ID_NO: 25) | CT (SEQ_ID_NO: 26) | |
| LINC01432- | GCAAGATCTCACCAGAAACCA | CAGTTTATTGTTTCACAG | RT-qPCR |
| tile5 | (SEQ_ID_NO: 27) | TTCTAGAGG | |
| | | (SEQ_ID_NO: 28) | |
| LINC01432- | TCTCACCAGAAACCACCCC | CAGTTCAGGCTGCTGTAA | RT-qPCR |
| tile6 | (SEQ_ID_NO: 29) | CA (SEQ_ID_NO: 30) | |
| LINC01432 | TGGAGATTTTGCCATT | | LNA ASO |
| LNA | (SEQ_ID_NO: 31) | | |
| LINC01432- | CTGATGCGCCTTACTC | | LNA ASO |
| 1 LNA | (SEQ_ID_NO: 35) | | |
| Control | AACACGTCTATACGC | | LNA ASO |
| LNA | (SEQ_ID_NO: 32) | | |
| CELF2 | GATGAACTCGTTACGC | | LNA ASO |
| | (SEQ_ID_NO: 33) | | |

### Multiplexed Fluorescent RNA In Situ Hybridization (Mfish).

RNAScope was performed as previously described, with some modifications using RNAscope 2.5 HD Reagent Kit Red assay combined with Immunohistochemistry (Advanced Cell Diagnostics [ACD], Catalog #323180 and #322372) according to manufacturer's instructions. Briefly, bone marrow aspirates or isolated tumors were applied to slides were baked in a dry air oven for one hour at 60° C., deparaffinized (Xylene for five minutes twice, followed by 100% ethanol for two minutes twice), hydrogen peroxide was applied for 10 minutes at room temperature, and co-detection target retrieval was performed using Steamer (BELLA) for twenty minutes and PBS-T washing. Slides were then incubated overnight with CUGBP2 (CELF2) antibody (Protein Tech, Cat #12921-1-AP) in a HybEz Slide Rack with damp humidifying paper and incubated overnight at 4° C. The next day, slides were washed in PBS-T then post-primary fixation was performed by submerging slides in 10% NBF for 30 minutes at room temperature. Slides were then washed with PBS-T and Protease Plus was added to each slide for 30 minutes at 40° C., then slides were washed with distilled water. Probes for LINC01432 (ACD Cat #878271) were then warmed at 40° C. and hybridized with specific oligonucleotide probes for 2 hours at 40° C. in HybEZ Humidifying System. RNA was then serial amplified and stained with Fast Red solution. Slides were blocked with co-detection blocker (ACD) for 15 minutes at 40° C. and washed with PBS-T. Secondary Alexa Fluor 488 antibody (Abcam, cat #ab150081) was applied for one hour at room temperature in the dark. Finally, slides were washed with PBS-T, counter stained with DAPI (Sigma, cat #D9542) for 30 seconds, and mounted with ProLong Gold Antifade Reagent (Invitrogen, cat #P36930). Slides were imaged on the EVOS M5000 Imaging System (Invitrogen).

Analysis of mFISH and IHC images was performed by comparing expression of LINC01432 or CELF2 between different cell lines or tissues and simultaneously verifying their cellular localization or intensity of expression. We first visualized our target RNA molecules using an EVOS M5000 imaging system and quantified targets with QuPath Software v0.5.1 to obtain cell-count per region and number of spots per cell data. We then applied multiplex analysis followed by the cell distribution analysis for detecting lncRNA spots in each cellular compartment.

### In Vitro Phenotypic Assays.

We used the ApoTox-Glo Triplex Assay (Promega, Madison, WI) to simultaneously measure viability, cytotoxicity, and apoptosis in the same sample. We seeded 20,000 cells/well of Control CRISPR/Cas9, LINC01432 knockdown CRISPR/Cas9, empty vector, LINC01432 overexpression, or wild-type RPMI 8226 cells in triplicate into a 96-well plate at 100 uls complete media per well. We began by first adding 20 ul of Viability/Cytoxicity reagent to all wells, and briefly mixing for ˜30 seconds. Plates were then placed in a 37° C. incubator for one hour. Next, we measured the intensity of fluorescence (relative fluorescence units) using 400Ex/505Em (viability) and 485Ex/520Em (Cytotoxicity) in Varioskan LUX microplate reader. To measure apoptosis, we next add 100 ul Caspase-Glo 3/7 reagent to the same wells and briefly mixed by orbital shaking ˜20 seconds, followed by incubation at room temp for 30 minutes. Luminescence (relative luminescence units) was then measured using Varioskan LUX microplate reader to detect caspase activation. A similar protocol was used to detect the proliferation of cells treated with Melphalan at increasing concentrations (Sigma Aldrich, St. Louis, MO, cat #M2011) using 10 uls Invitrogen alamarBlue HS Cell Viability Reagent in 90 μls of 25,000 cells with media (Waltham, MA, cat #A50100). Fluorescence was then measured using 560Ex/570Em in a microplate reader.

We measured apoptosis by isolating cells and assessing via flow cytometry using BD Horizon V450 AnnexinV (BD Biosciences, Franklin Lakes, NJ). We seeded 500,000 cells/well in a 6-well plate for 24 hours. Cells were then harvested, and the usual protocol was followed, per manufactures instructions. Briefly, cells were washed twice with PBS, then incubated V450 AnnexinV and Propidium Iodide (ThermoFisher) for fifteen minutes at room temperature in the dark. Apoptosis and DNA content was assessed on a flow cytometer machine (Novios, Becton Dickinson) by the Flow Cytometry Core of Siteman Cancer Center, Washington University in St. Louis. We collected a minimum of 50,000 cells per sample in triplicate. FlowJo Version10 (Becton Dickinson) was used to analyze data.

In Vivo Individual-Nucleotide Resolution Cross-Linking Immunoprecipitation (iCLIP).

Cells were seeded at a density of twenty million cells/150 mm dish. The next day, cells were washed with cold PBS and media volumes were adjusted to 10 ml/dish. Dishes were then uncovered and irradiated with 150 mJ/cm² of UVA (254 nm) in a crosslinker device (Stratalinker). Cells were then harvested and centrifuged at 2000 RPM at 4° C. for 5 minutes. Cell pellets were resuspended in 1 ml of NP-40 lysis buffer (20 mM Tris-HCl at pH 7.5, 100 mM KCl, 5 mM MgCl2, and 0.5% NP-40) with 1 μl protease inhibitor and 1 mM DTT, incubated on ice for ten minutes, and then centrifuged at 10,000 RPM for 15 minutes at 4° C. Supernatants were collected, 1 U/μl RNase T1 was added, then cell lysates were incubated at 22° C. for 30 minutes. Protein G Beads were resuspended in 100 μls NT2 buffer (50 mM Tris-HCl at pH 7.5, 150 mM NaCl,1 mM MgCl2, 0.05% NP-40) with 5 μg of respective antibodies, then rotated for one hour at room temperature. All antibodies and concentrations are listed in Table 3. Cell lysates were added to the beads and incubated for three hours at 4° C., the beads were washed with NT2 buffer, and then incubated with 20 units RNAse-free DNase I for 15 minutes at 37° C. in a thermomixer, shaking slowly. Protein kinase buffer (141 μls NP-40 lysis buffer, 0.1% SDS, 0.5 mg/ml Proteinase K) was then added and incubated for 15 minutes at 55° C. in a thermomixer, shaking at maximum speed. Supernatants were then collected, and RNA isolation was performed using a standard phenol:cholorform:isoamyl alcohol protocol. RNA was then reverse transcribed using SuperScript III First strand cDNA system, as per manufacturer's protocol (ThermoFisher) and primers tiling LINC01432 (Table 2) were used to detect LINC01432:protein binding.

TABLE 3
Antibodies

| Name | Vendor | Catalogue number | Application |
|---|---|---|---|
| CUGBP2 | Protein Tech | 12921-1-AP | RIP, CLIP, IF |
| C-myc | ABCAM | ab32072 | Western |
| Caspase-3 | Cell signaling | 96625 | Western |
| Actin | Cell signaling | 3700 | Western |
| P53, | Cell signaling | 2527S | Western |
| H2AX | Cell signaling | 9718S | Western |
| lgG | Cell signaling | 2729s | RIP, CLIP |

### In Vivo Myeloma Models.

All animal experiment protocols in this study were reviewed and approved by the Institutional Animal Care and Use Committee of Washington University in St. Louis. For subcutaneous injections, 2e⁵-1e⁷ cells (RPMI 8226 wild-type, U266B1 wild-type, Control CRISPR/Cas9, LINC01432 knockdown CRISPR/Cas9, empty vector, or LINC01432 overexpression) were subcutaneously injected NOD/SCID/γc^{-/-} (NSG) mice (N=5-10 per group). Resulting tumor size was quantified weekly via caliper measurements, comparing length×width×height×0.5. For post-analyses, subcutaneous tumor tissues were removed after sacrifice, formalin fixed, and paraffin embedded. This experiment was repeated twice.

### Data Sharing Statement

All RNA sequencing data is available at GEO under accession number GSE267013.

What is claimed is:

1. A composition for treatment of multiple myeloma in a patient in need, the composition comprising a LINC01432 inhibitor.

2. The composition of claim 1, wherein the LINC01432 inhibitor comprises an antisense oligonucleotide (ASO).

3. The composition of claim 2, wherein the ASO comprises an in vitro-locked nucleic acid GapmeR antisense oligonucleotide (LNA ASO).

4. The composition of claim 3, wherein the LNA ASO comprises SEQ_ID_NO:31.

5. The composition of claim 1, further comprising a multiple myeloma chemotherapy.

6. The composition of claim 5, wherein the chemotherapy is Melphalan.

7. The composition of claim 1, further comprising a CELF2 inhibitor.

8. The composition of claim 7, wherein the CELF2 inhibitor is a CELF2 LNA ASO.

9. A method to treat multiple myeloma in a patient in need, the method comprising administering a therapeutically effective amount of a LINC01432 inhibitor.

10. The method of claim 9, wherein the LINC01432 inhibitor comprises an antisense oligonucleotide (ASO).

11. The method of claim 10, wherein the ASO comprises an in vitro-locked nucleic acid GapmeR antisense oligonucleotide (LNA ASO).

12. The method of claim 11, wherein the LNA ASO comprises a sequence selected from the group consisting of SEQ_ID_NO:31 and SEQ_ID_NO:35.

13. The method of claim 9, further comprising administering a therapeutically effective amount a multiple myeloma chemotherapy.

14. The method of claim 5, wherein the multiple myeloma chemotherapy comprises Melphalan.

15. The method of claim 9, further comprising administering a therapeutically effective amount of a CELF2 inhibitor.

16. The method of claim 15, wherein the CELF2 inhibitor is a CELF2 LNA ASO.

17. A method of selecting a treatment for multiple myeloma in a patient in need, the method comprising:
quantifying an expression of a long non-coding RNA (lncRNA) comprising LINC01432;
determining an expression level of the lncRNA; and
selecting the treatment based on the expression level of the lncRNA, comprising:
administering a therapeutically effective amount of a multiple myeloma chemotherapy if the expression level of the lncRNA is below a threshold value; or
administering the therapeutically effective amount of the multiple myeloma chemotherapy and a therapeutically effective amount of a LINC01432 inhibitor if the lcRNA expression is above the threshold value.

18. The method of claim 17, wherein the LINC01432 inhibitor comprises an in vitro-locked nucleic acid GapmeR antisense oligonucleotide (LNA ASO) selected from the group consisting of SEQ_ID_NO:31 and SEQ_ID_NO:35.

19. The method of claim 18, wherein the threshold value comprises an expression level of about 6.42 fold higher relative to a healthy control expression level.

20. The method of claim 17, wherein the multiple myeloma chemotherapy comprises Melphalan.
